SEQUENCE LISTING (20 sequences)

SEQ ID NO: 1 - protein, 125 aa, synthetic construct
  REGION 1..125: note = synthetic
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 2 - protein, 8 aa, synthetic construct
  REGION 1..8: note = synthetic
GFTFSNFW

SEQ ID NO: 3 - protein, 8 aa, synthetic construct
  REGION 1..8: note = synthetic
MNQDGSEK

SEQ ID NO: 4 - protein, 18 aa, synthetic construct
  REGION 1..18: note = synthetic
ARDREYCISTSCYDDFDY

SEQ ID NO: 5 - protein, 124 aa, synthetic construct
  REGION 1..124: note = synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 6 - protein, 8 aa, synthetic construct
  REGION 1..8: note = synthetic
GFTFDDYS

SEQ ID NO: 7 - protein, 8 aa, synthetic construct
  REGION 1..8: note = synthetic
ISWNSGSK

SEQ ID NO: 8 - protein, 17 aa, synthetic construct
  REGION 1..17: note = synthetic
AKYGSGYGKFYHYGLDV

SEQ ID NO: 9 - protein, 124 aa, synthetic construct
  REGION 1..124: note = synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 10 - protein, 8 aa, synthetic construct
  REGION 1..8: note = synthetic
GFTFDDYS

SEQ ID NO: 11 - protein, 8 aa, synthetic construct
  REGION 1..8: note = synthetic
ISWNSGSI

SEQ ID NO: 12 - protein, 17 aa, synthetic construct
  REGION 1..17: note = synthetic
AKYGSGYGKFYYYGMDV

SEQ ID NO: 13 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 14 - protein, 6 aa, synthetic construct
  REGION 1..6: note = synthetic
QSISSY

SEQ ID NO: 15 - skipped

SEQ ID NO: 16 - protein, 10 aa, synthetic construct
  REGION 1..10: note = synthetic
QQSYSTPPIT

SEQ ID NO: 17 - protein, 451 aa, synthetic construct
  REGION 1..451: note = bsAb25441D and bsAb25442D anti-BCMA Heavy Chain
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 450 aa, synthetic construct
  REGION 1..450: note = bsAb25441D anti-CD3 Heavy Chain
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 19 - protein, 450 aa, synthetic construct
  REGION 1..450: note = bsAb25442D anti-CD3 Heavy Chain
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 20 - protein, 215 aa, synthetic construct
  REGION 1..215: note = bsAb25441D and bsAb25442D Common Light Chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0070998 A1
(43) Pub. Date: Mar. 12, 2026

(54) METHODS OF TREATING MULTIPLE MYELOMA WITH BCMA INHIBITORS IN COMBINATION WITH CD38 INHIBITORS

(71) Applicant: Regeneron Pharmaceuticals, Inc., Tarrytown, NY (US)

(72) Inventors: Glenn KROOG, New York, NY (US); Shawn M. SARKARIA, New York, NY (US)

(21) Appl. No.: 19/323,879

(22) Filed: Sep. 9, 2025

Related U.S. Application Data
(60) Provisional application No. 63/692,828, filed on Sep. 10, 2024.
(60) Provisional application No. 63/855,225, filed on Jul. 31, 2025.

(51) Int. Cl. C07K 16/28

(52) U.S. Cl. CPC C07K 16/2878; C07K 16/2809; C07K 16/2896; C07K 2317/31; C07K 2317/53; C07K 2317/565; C07K 2317/76

(57) ABSTRACT

The present disclosure provides methods for treating multiple myeloma. In certain embodiments, the present methods comprise administering to a subject in need thereof a BCMA inhibitor (e.g., a bispecific antibody or antigen-binding fragment thereof that bind to BCMA and CD3) in combination with a CD38 inhibitor (e.g., an anti-CD38 antibody). In certain embodiments, the subject has been previously treated with one or more anti-cancer therapies.

### CROSS-REFERENCE TO RELATED APPLICATIONS

This application claims the benefit under 35 USC § 119 (e) of US Provisional Application Nos.: 63/692,828, filed Sep. 10, 2024; and 63/855,225, filed Jul. 31, 2025, each of which is incorporated herein by reference in its entirety for all purposes.

### REFERENCE TO A SEQUENCE LISTING

This application incorporates by reference a computer readable Sequence Listing in ST.26 XML format, titled 11873US01_Sequence, created on Sep. 4, 2025 and containing 26,892 bytes.

### FIELD OF THE INVENTION

The present disclosure provides methods for treating refractory or relapsed multiple myeloma. In certain embodiments, the present methods comprise administering to a subject in need thereof a BCMA inhibitor (e.g., a bispecific antibody or antigen-binding fragment thereof that bind to BCMA and CD3) in combination with a CD38 inhibitor. In certain embodiments, the subject has been previously treated with one or more anti-cancer therapies.

### BACKGROUND

Multiple myeloma is a malignant neoplasm of plasma cells in the bone marrow associated with an overproduction of monoclonal (M)-protein often causing characteristic osteolytic lesions, anemia, renal failure, and hypercalcemia. Multiple myeloma patients who are refractory to multiple classes of therapies have reduced rates of overall survival (triple- and quad-refractory: 9.2 months, and penta-refractory: 5.6 months). Gandhi U. et al., Leukemia 33:2266-2275, 2013.

B-cell maturation antigen (BCMA), also known as TNFRSF17, or CD269, is a type III transmembrane protein lacking a signal peptide and containing a cysteine-rich extracellular domain. BCMA, along with closely related proteins, promotes B-cell survival at distinct stages of development. BCMA is expressed exclusively in B-cell lineage cells, particularly in the interfollicular region of the germinal center as well as on plasmablasts and differentiated plasma cells. BCMA is selectively induced during plasma cell differentiation, and is required for optimal survival of long-lived plasma cells in the bone marrow. In multiple myeloma, BCMA is widely expressed on malignant plasma cells at elevated levels, and BCMA expression is increased with progression from normal cells to active multiple myeloma. Lee et al., Br J. Haematol, 174 (6): 911-922, 2016. BCMA-directed bispecific antibodies have demonstrated encouraging efficacy results in early phase clinical studies for refractory or relapsed multiple myeloma patients. Although high response rates have been observed at optimized doses, the variability in depth and durability of responses remains poorly understood.

CD3 is a homodimeric or heterodimeric antigen expressed on T cells in association with the T cell receptor complex (TCR) and is required for T cell activation. Functional CD3 is formed from the dimeric association of two of four different chains: epsilon, zeta, delta and gamma. The CD3 dimeric arrangements include gamma/epsilon, delta/epsilon and zeta/zeta. Antibodies against CD3 have been shown to cluster CD3 on T cells, thereby causing T cell activation in a manner similar to the engagement of the TCR by peptide-loaded MHC molecules. Thus, anti-CD3 antibodies have been proposed for therapeutic purposes involving the activation of T cells. In addition, bispecific antibodies that are capable of binding CD3 and a target antigen have been proposed for therapeutic uses involving targeting T cell immune responses to tissues and cells expressing the target antigen.

Cluster of differentiation 38 (CD38) is a cell surface protein overexpressed in several types of cancer cells, including multiple myeloma. Anti-CD38 monoclonal antibodies, including daratumumab and isatuximab, have been used in combination therapies for the treatment of multiple myeloma.

Combination therapy for multiple myeloma has shown some benefits, but the disease remains incurable, and with each relapse, the progression-free survival shortens. Therefore, for patients with refractory or relapsed multiple myeloma, there is a compelling need for new therapeutics and methods of treatment.

### BRIEF SUMMARY OF THE DISCLOSURE

In one aspect, the present disclosure provides a method of treating relapsed or refractory multiple myeloma in a subject in need thereof, comprising administering to the subject a B-cell maturation antigen (BCMA) inhibitor, and a CD38 inhibitor.

In some embodiments, the CD38 inhibitor is an anti-CD38 antibody. In some embodiments, the CD38 inhibitor is daratumumab. In some embodiments, the CD38 inhibitor is isatuximab.

In some embodiments, the subject has not been treated with a prior anti-cancer therapy. In some embodiments, the subject has been previously treated with an anti-cancer therapy.

In some embodiments, the subject has been previously treated with two lines of anti-cancer therapy. In some embodiments, the subject has been previously treated with at least three lines of anti-cancer therapy. In some embodiments, the anti-cancer therapy is selected from an anti-CD38 antibody, an immunomodulatory imide (IMiD) drug and a PI. In some embodiments, the subject has been previously treated with a PI, an IMiD or a combination thereof. In some embodiments, the subject is double refractory to a PI and an IMID, or the combination of a PI and a IMiD.

In some embodiments, the relapsed or refractory multiple myeloma is multiple myeloma that has progressed during previous treatment; multiple myeloma that has progressed within 60 days after completion of therapy; or multiple myeloma that has had a less than 25% response to previous treatment.

In some embodiments, the subject is eligible for a stem cell transplant or has undergone a stem cell transplant. In some embodiments, the subject is not eligible for a stem cell transplant.

In some embodiments, the methods disclosed herein include administration of a premedication prior to the administration of the BCMA inhibitor or the CD38 inhibitor. In some embodiments, the premedication is an antihistamine, acetaminophen, dexamethasone, an IL6R inhibitor, or a combination thereof.

In some embodiments, the BCMA inhibitor and the CD38 inhibitor are administered in a dosing regimen comprising weekly (QW) administration of a full dose of the BCMA inhibitor and weekly (QW) administration of a dose of the CD38 inhibitor. In some embodiments, the dosing regimen comprises one or more 28-day cycles, wherein the BCMA inhibitor is administered weekly during each week of the cycle, and the CD38 inhibitor is administered weekly during each week of the cycle.

In some embodiments of the dosing regimen, the one or more 28-day cycles comprise one 28-day cycle. In some embodiments, the one or more 28-day cycles comprise two 28-day cycles. In some embodiments, the dosing regimen further comprises one or more additional 28-day cycles.

In some embodiments of the dosing regimen, the BCMA inhibitor is administered weekly (QW) or once every two weeks (Q2W) or once every four weeks (Q4W) during the one or more additional 28-day cycles, and the CD38 inhibitor is administered once every two weeks (Q2W) or once every four weeks (Q4W) during the one or more additional 28-day cycles.

In some embodiments, the CD38 inhibitor is daratumumab. In some embodiments, the CD38 inhibitor is isatuximab.

In some embodiments, the dosing regimen further comprises a step-up cycle preceding the one or more 28-day cycles, wherein the step-up cycle comprises administration of the BCMA inhibitor as monotherapy. In some embodiments, the BCMA inhibitor is administered during the step-up cycle at an initial dose, at an intermediate dose, wherein the intermediate dose is greater than the initial dose, and at a full dose, wherein the full dose is greater than the intermediate dose.

In some embodiments, the initial dose of the BCMA inhibitor is 0.5 mg to 10 mg. In some embodiments, the initial dose is about 5 mg. In some embodiments, the intermediate dose of the BCMA inhibitor is 20 mg to 30 mg. In some embodiments, the intermediate dose is about 25 mg. In some embodiments, the full dose of the BCMA inhibitor is 40 mg to 250 mg. In some embodiments, the full dose is about 50 mg, about 100 mg, about 150 mg, or about 200 mg.

In some embodiments, the dose of the CD38 inhibitor is 10 mg/kg to 25 mg/kg. In some embodiments, the dose of the CD38 inhibitor is about 16 mg/kg. In some embodiments, the dose of the CD38 inhibitor is 5 mg/kg to 15 mg/kg. In some embodiments, the dose of the CD38 inhibitor is about 10 mg/kg. In some embodiments, the dose of the CD38 inhibitor is about 1400 mg to about 2200 mg. In some embodiments, the dose of the CD38 inhibitor is about 1800 mg.

In some embodiments, the BCMA inhibitor and the CD38 inhibitor are administered instraveneously or subcutaneously to the subject.

In some embodiments, the CD38 inhibitor is administered subcutaneously in combination with hyaluronidase. In some embodiments, the dose of hyaluronidase is about 30,000 units.

In some embodiments, the BCMA inhibitor and the CD38 inhibitor are administered in separate formulations.

In some embodiments, the BCMA inhibitor is (i) an antibody or antigen-binding fragment thereof that binds specifically to BCMA, (ii) a bispecific antibody or antigen-binding fragment thereof comprising a first antigen-binding domain that specifically binds BCMA, and a second antigen-binding domain that specifically binds CD3, (iii) an antibody-drug-conjugate wherein the antibody binds specifically to BCMA, or (iv) a cell comprising a chimeric antigen receptor (CAR) that binds specifically to BCMA.

In some embodiments, the BCMA inhibitor is selected from the group consisting of AMG224, MEDI2228, 2A9-MICA, ALLO-605, alnuctamab, AMG-701, EM801, EMB-06, pacanalotamab, pavurutamab, linvoseltamab, vonsetamig, teclistamab, elranatamab, CM336, HBM7020, RO7297089, TQB2934, HPN217, ISB2001, IBI-3003, SAR445514, SIM0500, TNB-383B, WVT078, YKST02, balantamab mafodotin, ispectamab debotansine, LCAR-B38M, ciltacabtagene autoleucel, and idecabtagene vicleucel.

In some embodiments, the BCMA inhibitor is a bispecific antibody or antigen-binding fragment thereof comprising a first antigen-binding domain that specifically binds BCMA, and a second antigen-binding domain that specifically binds CD3.

In some embodiments, the first antigen-binding domain of the bispecific antibody or antigen-binding fragment thereof comprises three heavy chain complementarity determining regions, HCDR1, HCDR2 and HCDR3 comprised in a heavy chain variable region (HCVR) of SEQ ID NO: 1, and three light chain complementarity determining regions, LCDR1, LCDR2 and LCDR3 comprised in a light chain variable region (LCVR) of SEQ ID NO: 13. In some embodiments, the HCDR1, HCDR2 and HCDR3 comprise the amino acid sequences of SEQ ID NOs: 2, 3 and 4, respectively, and the LCDR1, LCDR2 and LCDR3, comprise the amino acid sequences of SEQ ID NOs: 14, 15 and 16, respectively. In some embodiments, the HCVR of the first antigen-binding domain comprises the amino acid sequence of SEQ ID NO: 1, and the LCVR of the first antigen-binding domain comprises the amino acid sequence of SEQ ID NO: 13.

In some embodiments, the second antigen-binding domain of the bispecific antibody or antigen-binding fragment thereof comprises three heavy chain complementarity determining regions, HCDR1, HCDR2 and HCDR3 comprised in a HCVR of SEQ ID NO: 5, and three light chain complementarity determining regions, LCDR1, LCDR2 and LCDR3 comprised in a LCVR of SEQ ID NO: 13. In some embodiments, the HCDR1, HCDR2 and HCDR3 comprise the amino acid sequences of SEQ ID NOs: 6, 7 and 8, respectively, and the LCDR1, LCDR2 and LCDR3 comprise the amino acid sequences of SEQ ID NOs: 14, 15 and 16, respectively. In some embodiments, the HCVR of the second antigen-binding domain comprises the amino acid sequence of SEQ ID NO: 5, and the LCVR of the first antigen-binding domain comprises the amino acid sequence of SEQ ID NO: 13.

In some embodiments, the second antigen-binding domain of the bispecific antibody or antigen-binding domain thereof comprises three heavy chain complementarity determining regions, HCDR1, HCDR2 and HCDR3 comprised in a HCVR of SEQ ID NO: 9, and three light chain complementarity determining regions, LCDR1, LCDR2 and LCDR3 comprised in a LCVR of SEQ ID NO: 13. In some embodiments, the HCDR1, HCDR2 and HCDR3 comprise the amino acid sequences of SEQ ID NOs: 10, 11 and 12, respectively, and the LCDR1, LCDR2 and LCDR3 comprise the amino acid sequences of SEQ ID NOs: 14, 15 and 16, respectively. In some embodiments, the HCVR of the second antigen-binding domain comprises the amino acid sequence of SEQ ID NO: 9, and the LCVR of the first antigen-binding domain comprises the amino acid sequence of SEQ ID NO: 13.

In some embodiments, the bispecific antibody comprises a human IgG heavy chain constant region. In some embodiments, the human IgG heavy chain constant region is isotype IgG1. In some embodiments, the human IgG heavy chain constant region is isotype IgG4.

In some embodiments, the bispecific antibody comprises a chimeric hinge that reduces Fcγ receptor binding relative to a wild-type hinge of the same isotype.

In some embodiments, the bispecific antibody comprises a first heavy chain and a second heavy chain, and wherein the first heavy chain or the second heavy chain, but not both, comprises a CH3 domain comprising a H435R (EU numbering) modification and a Y436F (EU numbering) modification.

In some embodiments, the bispecific antibody comprises a first heavy chain comprising the amino acid sequence of SEQ ID NO: 17, a second heavy chain comprising the amino acid sequence of SEQ ID NO: 18, and a common light chain paired with each of the first heavy chain and second heavy chain, respectively, comprising the amino acid sequence of SEQ ID NO: 20.

In some embodiments, the bispecific antibody comprises a first heavy chain comprising the amino acid sequence of SEQ ID NO: 17, a second heavy chain comprising the amino acid sequence of SEQ ID NO: 19, and a common light chain paired with each of the first heavy chain and second heavy chain, respectively, comprising the amino acid sequence of SEQ ID NO: 20.

In some embodiments, the bispecific antibody consists of two heavy chains and two lights chains interconnected by disulfide bonds, wherein each heavy chain comprises a HCVR and CH1, CH2 and CH3 heavy chain constant domains, and each light chain comprises a LCVR and a light chain constant (LC) domain.

In some embodiments, the BCMA inhibitor is linvoseltamab or vonsetamig.

In some embodiments, the methods disclosed herein include administering a third therapeutic agent or therapeutic regimen. In some embodiments, the third therapeutic agent or therapeutic regimen comprises a chemotherapeutic drug, a DNA alkylator, an immunomodulator, a second proteasome inhibitor, a histone deacetylase inhibitor, radiotherapy, a stem cell transplant, a different bispecific antibody that interacts with a different tumor cell surface antigen and a T cell or immune cell antigen, an antibody drug conjugate, a chimeric antigen receptor (CAR), a PD-1 antagonist, a PD-L1 antagonist, a CTLA-4 inhibitor, a LAG3 inhibitor, a CD28 agonist, an oncolytic virus, a cytokine, an IL4R inhibitor, an IL6R inhibitor, IL1R inhibitor, IL2, IL12, IL15, IL23, or combinations thereof.

Any of the various methods discussed above or herein can be reformatted as (i) a BCMA inhibitor for use in combination with a CD38 inhibitor (e.g., isatuximab or daratumumab) in a method for treating RRMM, or (ii) a CD38 inhibitor (e.g., isatuximab or daratumumab) for use in combination with a BCMA inhibitor in a method for treating RRMM, or (iii) use of a BCMA inhibitor in the manufacture of a medicament for treating RRMM in combination with a CD38 inhibitor (e.g., isatuximab or daratumumab), or (iv) use of a a CD38 inhibitor (e.g., isatuximab or daratumumab) in the manufacture of a medicament for treating RRMM in combination with a BCMA inhibitor, or (v) use of a combination of a BCMA inhibitor and a CD38 inhibitor (e.g., isatuximab or daratumumab) in the manufacture of a medicament for treating RRMM (e.g., where the two agents are formulated for separate administration).

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

Other embodiments will become apparent from a review of the ensuing detailed description.

### BRIEF DESCRIPTION OF THE DRAWINGS

FIG. 1 illustrates an exemplary dosing schedule, as discussed in Example 2 for treatment of refractory multiple myeloma with an anti-BCMA x anti-CD3 bispecific antibody in combination with a CD38 inhibitor (e.g., daratumumab).

FIG. 2 illustrates an exemplary dosing schedule, as discussed in Example 3 for treatment of refractory multiple myeloma with an anti-BCMA x anti-CD3 bispecific antibody in combination with a CD38 inhibitor (e.g., isatuximab).

### DETAILED DESCRIPTION

It is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the subject matter of the present disclosure, the preferred methods and materials are now described. All patents, applications and non-patent publications mentioned in this specification are incorporated herein by reference in their entireties.

### Definitions

The expression “CD3,” as used herein, refers to an antigen which is expressed on T cells as part of the multimolecular T cell receptor (TCR) and which consists of a homodimer or heterodimer formed from the association of two of four receptor chains: CD3-epsilon, CD3-delta, CD3-zeta, and CD3-gamma. All references to proteins, polypeptides and protein fragments herein are intended to refer to the human version of the respective protein, polypeptide or protein fragment unless explicitly specified as being from a non-human species. Thus, the expression “CD3” means human CD3 unless specified as being from a non-human species, e.g., “mouse CD3,” “monkey CD3,” etc.

As used herein, “an antibody that binds CD3” or an “anti-CD3 antibody” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). The antibodies and antigen-binding fragments of the present disclosure may bind soluble CD3 and/or cell surface expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

As used herein, the expression “cell surface-expressed CD3” means one or more CD3 protein(s) that is/are expressed on the surface of a cell in vitro or in vivo, such that at least a portion of a CD3 protein is exposed to the extracellular side of the cell membrane and is accessible to an antigen-binding portion of an antibody. “Cell surface-expressed CD3” includes CD3 proteins contained within the context of a functional T cell receptor in the membrane of a cell. The expression “cell surface-expressed CD3” includes CD3 protein expressed as part of a homodimer or heterodimer on the surface of a cell (e.g., gamma/epsilon, delta/epsilon, and zeta/zeta CD3 dimers). The expression, “cell surface-expressed CD3” also includes a CD3 chain (e.g., CD3-epsilon, CD3-delta or CD3-gamma) that is expressed by itself, without other CD3 chain types, on the surface of a cell. A “cell surface-expressed CD3” can comprise or consist of a CD3 protein expressed on the surface of a cell which normally expresses CD3 protein. Alternatively, “cell surface-expressed CD3” can comprise or consist of CD3 protein expressed on the surface of a cell that normally does not express human CD3 on its surface but has been artificially engineered to express CD3 on its surface.

The expression “BCMA,” as used herein, refers to B-cell maturation antigen. BCMA (also known as TNFRSF17 and CD269) is a cell surface protein expressed on malignant plasma cells, and plays a central role in regulating B cell maturation and differentiation into immunoglobulin-producing plasma cells. The amino acid sequence of human BCMA can be found in GenBank accession number NP_001183.2.

The term “BCMA inhibitor,” as used herein refers to a molecule or a cell comprising such a molecule, that binds specifically to BCMA and inhibits its activity. In the context of the disclosure, the BCMA inhibitor includes (i) an antibody or antigen-binding fragment thereof that binds specifically to BCMA, (ii) a bispecific antibody or antigen-binding fragment thereof comprising a first antigen-binding domain that specifically binds BCMA, and a second antigen-binding domain that specifically binds CD3, (iii) an antibody-drug-conjugate wherein the antibody binds specifically to BCMA, or (iv) a cell comprising a chimeric antigen receptor (CAR) that binds specifically to BCMA. Examples of BCMA inhibitors include, but are not limited to, AMG224, MEDI2228, 2A9-MICA, ALLO-605, alnuctamab, AMG-701, EM801, EMB-06, pacanalotamab, pavurutamab, linvoseltamab, vonsetamig, teclistamab, elranatamab, CM336, HBM7020, RO7297089, TQB2934, HPN217, ISB2001, IBI-3003, SAR445514, SIM0500, TNB-383B, WVT078, YKST02, balantamab mafodotin, ispectamab debotansine, LCAR-B38M, ciltacabtagene autoleucel, and idecabtagene vicleucel.

The expression “CD38,” as used herein, refers to cluster of differentiation 38 (CD38). CD38 is a cell surface protein expressed on immune cells, most frequently on plasma B cells. The amino acid sequence of human CD38 can be found in GenBank accession number NP_001766.2.

A “plasma cell” is a differentiated B-lymphocyte capable of secreting antibodies.

As used herein, “refractory multiple myeloma” refers to multiple myeloma disease progression during treatment or disease progression within 60 days after completion of therapy, or less than 25% response to therapy. Prior therapies to which the disease was refractory can include treatment with IMiDs, proteasome inhibitors, anti-CD38 antibodies, or a combination thereof. IMiDs can include thalidomide, lenalidomide, and/or pomalidomide. Proteasome inhibitors can include bortezomib, carfilzomib, and/or ixazomib. Anti-CD38 antibodies can include daratumumab and/or isatuximab.

References to “end-organ damage or a myeloma-defining event that can be attributed to an underlying plasma cell proliferative disorder” for defining symptomatic multiple myeloma include one or more of the following:
(i) hypercalcemia, wherein serum calcium is >11 mg/dL;
(ii) renal insufficiency, wherein serum creatinine is >177 μmol/L;
(iii) anemia with a hemoglobin value ≥2 g/dL below the lower limit of normal, or a hemoglobin value of <10 g/dL;
(iv) more than one myeloma-related lesion on advanced imaging, including magnetic resonance imaging (MRI), positron emission tomography/computed tomography (PET/CT), or whole-body low dose computed tomography (WBLDCT);
(v) clonal bone marrow plasma cells ≥60%; or
(vi) involved/uninvolved serum free light chain (FLC) ratio of 100 or more.

As used herein, “an antibody that binds BCMA” or an “anti-BCMA antibody” includes antibodies and antigen-binding fragments thereof that specifically recognize BCMA.

The term “antigen-binding molecule” includes antibodies and antigen-binding fragments of antibodies, including, e.g., bispecific antibodies.

The term “antibody”, as used herein, means any antigen-binding molecule or molecular complex comprising at least one complementarity determining region (CDR) that specifically binds to or interacts with a particular antigen (e.g., BCMA, CD3, or CD38). The term “antibody” includes immunoglobulin molecules comprising four polypeptide chains, two heavy (H) chains and two light (L) chains inter-connected by disulfide bonds, as well as multimers thereof (e.g., IgM). The term “antibody” also includes immunoglobulin molecules consisting of four polypeptide chains, two heavy (H) chains and two light (L) chains inter-connected by disulfide bonds (e.g., IgG). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or VL) and a light chain constant region. The light chain constant region comprises one domain (CL1). The V_{H} and VL regions can be further subdivided into regions of hypervariability, termed complementarity determining regions (CDRs), interspersed with regions that are more conserved, termed framework regions (FR). Each V_{H} and VL is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-BCMA antibody or anti-CD3 antibody (or antigen-binding portion thereof) may be identical to the human germline sequences, or may be naturally or artificially modified. An amino acid consensus sequence may be defined based on a side-by-side analysis of two or more CDRs. In some cases, the “antibody” is an immunoglobulin molecule consisting of four polypeptide chains, two heavy (H) chains and two light (L) chains inter-connected by disulfide bonds in which the two heavy chains are both of isotype IgG1 or the two heavy chains are both of isotype IgG4. In a bispecific antibody, the two light (L) chains may be identical (common light chain) even though the two heavy chains contain distinct HCVRs. In some embodiments, the anti-BCMA x anti-CD3 bispecific antibody referenced herein is linvoseltamab.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′) 2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

In certain embodiments of the disclosure, the anti-BCMA monospecific antibodies or anti-BCMA x anti-CD3 bispecific antibodies of the disclosure are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies of the disclosure may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, C_{H}2 or C_{H}3 region which may be desirable, for example, in production, to improve the yield of the desired antibody form.

The antibodies of the disclosure may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The anti-BCMA x anti-CD3 antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure also includes anti-BCMA x anti-CD3 antibodies comprising variants of any of the HCVR, LCVR, and/or CDR amino acid sequences disclosed herein having one or more conservative substitutions. For example, the present disclosure includes anti-BCMA x anti-CD3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth herein. In some cases, the antibodies share 95%, 96%, 97%, 98% or 99% amino acid sequence identity with the HCVR(s) and/or LCVR or heavy chain(s) and/or light chain discussed herein.

As used herein, the term “binding” in the context of the binding of an antibody, immunoglobulin, antibody-binding fragment, or Fc-containing protein to either, e.g., a predetermined antigen, such as a cell surface protein or fragment thereof, typically refers to an interaction or association between a minimum of two entities or molecular structures, such as an antibody-antigen interaction. For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used, and FACS data correlates well with other methods such as radioligand competition binding and SPR (Benedict, C A, J Immunol Methods. 1997, 201 (2): 223-31; Geuijen, C A, et al. J Immunol Methods. 2005, 302 (1-2): 68-77). Accordingly, the antibody or antigen-binding protein of the disclosure binds to the predetermined antigen or cell surface molecule (receptor) having an affinity corresponding to a K_{D} value that is at least ten-fold lower than its affinity for binding to a non-specific antigen (e.g., BSA, casein). According to the present disclosure, the affinity of an antibody corresponding to a K_{D} value that is equal to or less than ten-fold lower than a non-specific antigen may be considered non-detectable binding, however such an antibody may be paired with a second antigen binding arm for the production of a bispecific antibody of the disclosure.

### Methods of Treating Relapsed or Refractory Multiple Myeloma

Multiple myeloma (MM) is a malignancy associated with the clonal proliferation of plasma cells. Multiple myeloma accounts for 1% of neoplastic diseases and is the second most common hematological malignancy in high-income countries. The incidence of MM is 4.5 to 6 per 100,000 per year, and the median age of diagnosis is about 70 years. Incidence is higher in western Europe, North America, and Australia than in Asia and sub-Saharan Africa, possibly because of variation in diagnosis. From 1990 to 2016, there was a 126% increase in global incidence of MM, owing to population growth, an aging world population, and increased age-specific incidence rates (van de Donk, 2021). MM is incurable and has a 5-year overall survival of approximately 50%. Although there are effective drugs for newly diagnosed patients with MM that provide a median remission period of up to 60 months, the cancer eventually progresses following a relapse and remitting course despite treatment. With each subsequent relapse, the progression-free survival (PFS) shortens, and by the third line of therapy, the median PFS is less than 1 year.

Multiple myeloma (MM) is characterized by the clonal proliferation of plasma cells. A diagnosis of MM requires the presence of at least 10% clonal plasma cells on bone marrow biopsy analysis and the presence of a monoclonal protein in either the serum or urine. In the case of true non-secretory plasma cell myeloma, the diagnosis is made with a bone marrow biopsy demonstrating at least 30% monoclonal plasma cells or a biopsy-proven plasmacytoma.

In any of the methods discussed above or herein, the subject may have relapsed or refractory multiple myeloma. In some cases, the subject has relapsed or refractory multiple myeloma following one or more (e.g., two or more, three or more, four or more, or five or more) prior systemic treatments, including any one or more of the previous treatments discussed above or herein. In certain embodiments, the subject is eligible for or has received a stem cell transplant. In other embodiments, the subject is not eligible for a stem cell transplant. In any of the methods discussed above or herein, the subject is at least triple-refractory to prior therapies (i.e., progressed after at least three prior lines of therapy). In some cases, the subject is quad-refractory to prior therapies. In some cases, the subject is penta-refractory to prior therapies. Refractory disease is defined as disease progression during treatment or disease progression within 60 days after completion of therapy, or less than 25% response to therapy. Prior therapies to which the disease was refractory can include treatment with IMiDs, proteasome inhibitors, anti-CD38 antibodies, or a combination thereof. IMiDs can include thalidomide, lenalidomide, and/or pomalidomide. Proteasome inhibitors can include bortezomib, carfilzomib, and/or ixazomib. Anti-CD38 antibodies can include daratumumab and/or isatuximab.

Symptomatic myeloma is defined by evidence of myeloma-related organ or tissue involvement, which includes hypercalcemia, or renal insufficiency, or anemia, or bone disease, or the presence of 60% or more marrow plasmacytosis, or an involved/uninvolved serum free-light chain (FLC) ratio of 100 or more, or more than 1 myeloma-related lesion on advanced imaging (positron emission tomography with embedded computed tomography [PET/CT] or magnetic resonance imaging [MRI]). Response to treatment can be assessed by monitoring levels of serum monoclonal protein (termed “M-protein”), urine monoclonal immunoglobulin light chain (termed Bence-Jones protein), and clonal plasma cells on bone marrow biopsy. In some cases, the subjects have bone-associated plamacytomas.

The present disclosure includes methods for treating refractory or relapsed multiple myleoma in subjects. The methods according to this aspect of the disclosure comprise administering a BCMA inhibitor, in combination with a CD38 inhibitor, to a subject in need thereof. As used herein, the terms “treat”, “treating”, or the like, mean to alleviate symptoms, eliminate the causation of symptoms either on a temporary or permanent basis, to delay or inhibit the proliferation of plasma cells producing abnormal light chains or other M-proteins. As used herein, the expression “a subject in need thereof” means a human or non-human animal that exhibits one or more indicia of symptomic multiple myeloma as discussed herein.

The methods of the present disclosure, according to certain embodiments, comprise administering to a subject a BCMA inhibitor (e.g., a bispecific anti-BCMA/anti-CD3 antibody or antigen-binding fragment thereof), in combination with a CD38 inhibitor. In certain embodiments, the combination may be administered in further combination with an additional therapy including a chemotherapeutic agent, radiation, surgery, and an autologous stem-cell transplant. As used herein, the phrase “in combination with” means that the BCMA inhibitor and the CD38 inhibitor are administered to the subject in a time frame in which the combination can exert an overlapping effect. In certain embodiments, the BCMA inhibitor and the CD38 inhibitior are administered in separate formulations. In certain embodiments, the BCMA inhibitor and the CD38 inhibitor are administered in the same formulation.

In certain embodiments, the methods of the present disclosure comprise administering to a subject in need thereof a BCMA inhibitor (e.g., a bispecific anti-BCMA/anti-CD3 antibody or antigen-binding fragment thereof) in combination with a CD38 inhibitor, which results in minimal residual disease (one of four categories, which also require a complete response as defined), a stringent complete response (sCR), a complete response (CR), a very good partial response (VGPR), a partial response (PR), a minimal response (MR), or stable disease (SD), as defined by the International Myeloma Working Group (IMWG) response criteria, shown in Table 1, below.

TABLE 1
IMWG Response Criteria Including MRD Criteria

| Category | Criteria |
|---|---|
| Sustained | MRD-negativity in the marrow (NGF or NGS, or both) and by imaging as defined below, |
| MRD- | confirmed minimum of 1 year apart. Subsequent evaluations can be used to further specify |
| negative | the duration of negativity (e.g., MRD-negative at 5 years) |
| Flow MRD- | Absence of phenotypically aberrant clonal plasma cells by next generation flow (NGF) on |
| negative | bone marrow aspirates using the EuroFlow standard operation procedure for MRD detection |
| | in multiple myeloma (or validated equivalent method) with a minimum sensitivity of 1 in 10⁵ |
| | nucleated cells or higher |
| Sequencing | Absence of clonal plasma cells by next generation sequencing (NGS) on bone marrow |
| MRD- | aspirate in which presence of a clone is defined as less than two identical sequencing reads |
| negative | obtained after DNA sequencing of bone marrow aspirates using the LymphoSIGHT platform |
| | (or validated equivalent method) with a minimum sensitivity of 1 in 10⁵ nucleated cells or |
| | higher |
| Imaging | MRD-negativity as defined by NGF or NGS plus disappearance of every area of increased |
| plus MRD- | tracer uptake found at baseline or a preceding PET/CT or decrease to less mediastinal blood |
| negative | pool SUV or decrease to less than that of surrounding normal tissue |
| Stringent | Complete response as defined below plus normal free light chain (FLC) ratio and absence of |
| complete | clonal cells in bone marrow biopsy by immunohistochemistry (κ/λ ratio ≤4:1 or ≥1:2 for κ and |
| response | λ patients, respectively, after counting ≥100 plasma cells) |
| Complete | Negative immunofixation on the serum and urine and disappearance of any soft tissue |
| response | plasmacytomas and <5% plasma cells in bone marrow aspirates |
| Very good | Serum and urine M-protein detectable by immunofixation but not on electrophoresis or ≥90% |
| partial | reduction in serum M-protein plus urine M-protein level <100 mg per 24 h |
| response | |
| Partial | ≥50% reduction of serum M-protein plus reduction in 24 h urinary M-protein by ≥90% or |
| response | to <200 mg per 24 h; |
| | If the serum and urine M-protein are unmeasurable, a ≥50% decrease in the difference |
| | between involved and uninvolved FLC levels is required in place of the M-protein criteria; |
| | If serum and urine M-protein are unmeasurable, and serum-free light assay is also |
| | unmeasurable, ≥50% reduction in plasma cells is required in place of M-protein, provided |
| | baseline bone marrow plasma-cell percentage was ≥30%. In addition to these criteria, if |
| | present at baseline, a ≥50% reduction in the size (SPD) of soft tissue plasmacytomas is also |
| | required |
| Minimal | ≥25% but ≤49% reduction of serum M-protein and reduction in 24-h urine M-protein by 50- |
| response | 89%. In addition to the above listed criteria, if present at baseline, a 25% to 49% reduction |
| | in the size (SPD) of soft tissue plasmacytomas is also required |
| Stable | Not recommended for use as an indicator of response; stability of disease is best described |
| disease | by providing the time-to-progression estimates. Not meeting criteria for complete response, |
| | very good partial response, partial response, minimal response, or progressive disease |
| Progressive | Any one or more of the following criteria: |
| disease | Increase of 25% from lowest confirmed response value in one or more of the following |
| | criteria: |
| | Serum M-protein (absolute increase must be ≥0.5 g/dL); |
| | Serum M-protein increase ≥1 g/dL, if the lowest M component was ≥5 g/dL; |
| | Urine M-protein (absolute increase must be ≥200 mg/24 h); |
| | In patients without measurable serum and urine M-protein levels, the difference |
| | between involved and uninvolved FLC levels (absolute increase must be >10 mg/dL); |
| | In patients without measurable serum and urine M-protein levels and without |
| | measurable involved FLC levels, bone marrow plasma-cell percentage irrespective of |
| | Baseline status (absolute increase must be ≥10%); |
| | Appearance of a new lesion(s), ≥50% increase from nadir in SPD of >1 lesion, or ≥50% |
| | increase in the longest diameter of a previous lesion >1 cm in short axis; |
| | ≥50% increase in circulating plasma cells (minimum of 200 cells per μL) if this is the |
| | only measure of disease |

In certain embodiments, the methods of the present disclosure are used to treat a patient with RRMM to yield minimal-residual disease (MRD) negativity or sustained MRD negativity (e.g., for more than 1 month, more than 3 months, more than 6 months, more than 12 months, more than 18 months, more than 24 months, more than 36 months, more than 48 months, more than 5 years, or more than 10 years). Minimum residual disease (MRD) refers to small numbers of cancer cells that remain in the patient during or after treatment, wherein the patient may or may not show symptoms or signs of the disease. Such residual cancer cells, if not eliminated, frequently lead to relapse of the disease. The present disclosure includes methods to inhibit and/or eliminate residual cancer cells in a patient upon MRD testing. MRD may be assayed according to methods known in the art (e.g., MRD flow cytometry).

In certain embodiments, the administration of the BCMA inhibitor in combination with a CD38 inhibitor increases duration of survival of the subject, e.g., increases duration of survival by more than 1 month, more than 3 months, more than 6 months, more than 12 months, more than 18 months, more than 24 months, more than 36 months, more than 48 months, more than 5 years, more than 6 years, more than 7 years, more than 8 years, more than 9 years, or more than 10 years, relative to an untreated subject or a subject treated with either agent as monotherapy. In certain embodiments, administration of the BCMA inhibitor in combination with a CD38 inhibitor increases progression-free survival or overall survival. In certain embodiments, administration of the BCMA inhibitor in combination with a CD38 inhibitor increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject or a subject treated with either agent as monotherapy.

### Bispecific Anti-BCMA x Anti-CD3 Antibodies

According to certain exemplary embodiments of the present disclosure, the methods comprise administering a BCMA inhibitor in combination with administration of a CD38 inhibitor, wherein the BCMA inhibitor is a bispecific antibody or antigen-binding fragment thereof that specifically binds BCMA and CD3. The antibodies and fragments may be referred to herein as, e.g., “anti-BCMA/anti-CD3,” or “anti-BCMA x CD3” or “BCMA x CD3” bispecific antibodies or antigen-binding fragments thereof, or other similar terminology. One example of an anti-BCMA x CD3 bispecific antibody is REGN5458.

As used herein, the expression “bispecific antibody” refers to an immunoglobulin protein comprising at least a first antigen-binding domain and a second antigen-binding domain. In the context of the present disclosure, the first antigen-binding domain specifically binds a first antigen (e.g., BCMA), and the second antigen-binding domain specifically binds a second, distinct antigen (e.g., CD3). Each antigen-binding domain of a bispecific antibody comprises a heavy chain variable domain (HCVR) and a light chain variable domain (LCVR), each comprising three complementarity determining regions (CDRs). In the context of a bispecific antibody, the CDRs of the first antigen-binding domain may be designated with the prefix “A” and the CDRs of the second antigen-binding domain may be designated with the prefix “B”. Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain are each connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present disclosure, the multimerizing component is an Fc portion of an immunoglobulin (comprising a C_{H2}-C_{H3} domain), e.g., an Fc domain of an IgG selected from the isotypes IgG1, IgG2, IgG3, and IgG4, as well as any allotype within each isotype group.

Bispecific antibodies of the present disclosure typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antibodies of the present disclosure. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antibody. Specific exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats).

In the context of bispecific antibodies of the present disclosure, Fc domains may comprise one or more amino acid changes (e.g., insertions, deletions or substitutions) as compared to the wild-type, naturally occurring version of the Fc domain. For example, the disclosure includes bispecific antibodies comprising one or more modifications in the Fc domain that results in a modified Fc domain having a modified binding interaction (e.g., enhanced or diminished) between Fc and FcRn. In one embodiment, the bispecific antibody comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications are disclosed in US Patent Publication No. 20150266966, incorporated herein in its entirety.

The present disclosure also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antibodies set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antibodies set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains or chimeric heavy chain constant regions that can be included in any of the antibodies of the present disclosure are described in US Patent Publication No. 20140243504, which is herein incorporated in its entirety. Chimeric Fc domains and chimeric heavy chain constant regions having these general structural arrangements, and variants thereof, can have altered Fc receptor binding, which in turn affects Fc effector function.

According to certain exemplary embodiments of the present disclosure, the bispecific anti-BCMA/anti-CD3 antibody, or antigen-binding fragment thereof comprises heavy chain variable regions (A-HCVR and B-HCVR), light chain variable regions (A-LCVR and B-LCVR), and/or complementarity determining regions (CDRs) comprising any of the amino acid sequences of the bispecific anti-BCMA/anti-CD3 antibodies as set forth in WO 2020/018820. In certain exemplary embodiments, the bispecific anti-BCMA/anti-CD3 antibody or antigen-binding fragment thereof that can be used in the context of the methods of the present disclosure comprises: (a) a first antigen-binding arm that specifically binds BCMA comprising the heavy chain complementarity determining regions (A-HCDR1, A-HCDR2 and A-HCDR3) of a heavy chain variable region (A-HCVR) comprising the amino acid sequence of SEQ ID NO: 1 and the light chain complementarity determining regions (A-LCDR1, A-LCDR2 and A-LCDR3) of a light chain variable region (A-LCVR) comprising the amino acid sequence of SEQ ID NO: 13; and (b) a second antigen-binding arm that specifically binds CD3 comprising the heavy chain CDRs (B-HCDR1, B-HCDR2 and B-HCDR3) of a HCVR (B-HCVR) comprising an amino acid sequence of SEQ ID NO: 5, and the light chain CDRs (B-LCDR1, B-LCDR2 and B-LCDR3) of a LCVR (B-LCVR) comprising the amino acid sequence of SEQ ID NO: 13. According to certain embodiments, the A-HCDR1 comprises the amino acid sequence of SEQ ID NO: 2; the A-HCDR2 comprises the amino acid sequence of SEQ ID NO: 3; the A-HCDR3 comprises the amino acid sequence of SEQ ID NO: 4; the A-LCDR1 comprises the amino acid sequence of SEQ ID NO: 14; the A-LCDR2 comprises the amino acid sequence of SEQ ID NO: 15; the A-LCDR3 comprises the amino acid sequence of SEQ ID NO: 16; the B-HCDR1 comprises the amino acid sequence of SEQ ID NO: 6; the B-HCDR2 comprises the amino acid sequence of SEQ ID NO: 7; and the B-HCDR3 comprises the amino acid sequence of SEQ ID NO: 8; and the B-LCDR1 comprises the amino acid sequence of SEQ ID NO: 14; the B-LCDR2 comprises the amino acid sequence of SEQ ID NO: 15; the B-LCDR3 comprises the amino acid sequence of SEQ ID NO: 16. In yet other embodiments, the bispecific anti-BCMA/anti-CD3 antibody or antigen-binding fragment thereof comprises: (a) a first antigen-binding arm comprising a HCVR (A-HCVR) comprising SEQ ID NO: 1 and a LCVR (A-LCVR) comprising SEQ ID NO: 13; and (b) a second antigen-binding arm comprising a HCVR (B-HCVR) comprising SEQ ID NO: 5, and a LCVR (B-LCVR) comprising SEQ ID NO: 13. In certain exemplary embodiments, the bispecific anti-BCMA x CD3 antibody comprises a BCMA-binding arm comprising a heavy chain comprising the amino acid sequence of SEQ ID NO: 17 and a light chain comprising the amino acid sequence of SEQ ID NO: 20, and a CD3-binding arm comprising a heavy chain comprising the amino acid sequence of SEQ ID NO: 18 and a light chain comprising the amino acid sequence of SEQ ID NO: 20. In some cases, the C-terminal lysine residue of SEQ ID NO: 18 or SEQ ID NO: 19 may be absent.

In certain exemplary embodiments, the bispecific anti-BCMA/anti-CD3 antibody or antigen-binding fragment thereof that can be used in the context of the methods of the present disclosure comprises: (a) a first antigen-binding arm that specifically binds BCMA comprising the heavy chain complementarity determining regions (A-HCDR1, A-HCDR2 and A-HCDR3) of a heavy chain variable region (A-HCVR) comprising the amino acid sequence of SEQ ID NO: 1 and the light chain complementarity determining regions (A-LCDR1, A-LCDR2 and A-LCDR3) of a light chain variable region (A-LCVR) comprising the amino acid sequence of SEQ ID NO: 13; and (b) a second antigen-binding arm that specifically binds CD3 comprising the heavy chain CDRs (B-HCDR1, B-HCDR2 and B-HCDR3) of a HCVR (B-HCVR) comprising an amino acid sequence of SEQ ID NO: 9, and the light chain CDRs (B-LCDR1, B-LCDR2 and B-LCDR3) of a LCVR (B-LCVR) comprising the amino acid sequence of SEQ ID NO: 13. According to certain embodiments, the A-HCDR1 comprises the amino acid sequence of SEQ ID NO: 2; the A-HCDR2 comprises the amino acid sequence of SEQ ID NO: 3; the A-HCDR3 comprises the amino acid sequence of SEQ ID NO: 4; the A-LCDR1 comprises the amino acid sequence of SEQ ID NO: 14; the A-LCDR2 comprises the amino acid sequence of SEQ ID NO: 15; the A-LCDR3 comprises the amino acid sequence of SEQ ID NO: 16; the B-HCDR1 comprises the amino acid sequence of SEQ ID NO: 10; the B-HCDR2 comprises the amino acid sequence of SEQ ID NO: 11; and the B-HCDR3 comprises the amino acid sequence of SEQ ID NO: 12; and the B-LCDR1 comprises the amino acid sequence of SEQ ID NO: 14; the B-LCDR2 comprises the amino acid sequence of SEQ ID NO: 15; the B-LCDR3 comprises the amino acid sequence of SEQ ID NO: 16. In yet other embodiments, the bispecific anti-BCMA/anti-CD3 antibody or antigen-binding fragment thereof comprises: (a) a first antigen-binding arm comprising a HCVR (A-HCVR) comprising SEQ ID NO: 1 and a LCVR (A-LCVR) comprising SEQ ID NO: 13; and (b) a second antigen-binding arm comprising a HCVR (B-HCVR) comprising SEQ ID NO: 9, and a LCVR (B-LCVR) comprising SEQ ID NO: 13. In certain exemplary embodiments, the bispecific anti-BCMA x CD3 antibody comprises a BCMA-binding arm comprising a heavy chain comprising the amino acid sequence of SEQ ID NO: 17 and a light chain comprising the amino acid sequence of SEQ ID NO: 20, and a CD3-binding arm comprising a heavy chain comprising the amino acid sequence of SEQ ID NO: 19 and a light chain comprising the amino acid sequence of SEQ ID NO: 20.

In certain embodiments, the methods comprise administering a bispecific BCMA X CD3 antibody in combination with a proteasome inhibitor, wherein the bispecific BCMA x CD3 antibody is selected from the group consisting of alnuctamab, AMG-701, EM801, EMB-06, pacanalotamab, pavurutamab, linvoseltamab (REGN5458), vonsetamig (REGN5459), teclistamab, elranatamab, CM336, HBM7020, RO7297089, TQB2934, HPN217, ISB2001, IBI-3003, SAR445514, SIM0500, TNB-383B, WVT078, and YKST02.

The methods of the present disclosure also encompass use of a bioequivalent to the bispecific antibodies discussed herein. The term “bioequivalent”, as used herein, refers to antibodies or fragments thereof that are pharmaceutical equivalents or pharmaceutical alternatives whose rate and/or extent of absorption do not show a significant difference with that of the bispecific antibodies discussed above when administered at the same molar dose under similar experimental conditions, either single dose or multiple dose. In the context of the disclosure, the term refers to antigen-binding proteins that bind to BCMA and CD3, which do not have clinically meaningful differences to the bispecific antibodies discussed herein in their safety, purity and/or potency.

### CD38 Inhibitors

Exemplary methods of the present disclosure comprise administering a BCMA inhibitor (e.g., a bispecific antibody that specifically binds BCMA and CD3 or an antigen-binding fragment thereof), in combination with a CD38 inhibitor. Cluster of differentiation 38 (CD38) is a cell surface protein with functions in cell adhesion and signalling. Plasma cells and some subsets of nonmalignant immune effector cells express CD38, which has generated interest in how anti-CD38 antibody therapies alter the immune microenvironment.

CD38 inhibitors can be, for example, an anti-CD38 antibody or an antigen-binding fragment thereof. Some examples of CD38 inhibitors include isatuximab (Sarclisa®) and daratumumab (Darzalex®). Isatuximab is an anti-CD38 monoclonal antibody with anti-myeloma activity that is approved for intravenous (IV) use in combination with pomalidomide and dexamethasone or carfilzomib and dexamethasone for the treatment of relapsed/refractory multiple myeloma (RRMM). Daratumumab is a fully human anti-CD38 monoclonal antibody with anti-myeloma activity in patients with relapsed/refractory multiple myeloma. In a phase 2 daratumumab monotherapy trial, 106 patients with RRMM who had previously received a median of 5 lines of therapy were found to have a 29% objective response rate with 12.2% achieving a greater than or equal to very good partial response. The median duration of response was 7.4 months and progression-free survival was 3.7 months. Anti-CD38 antibodies in general are encompassed within the scope of the present disclosure, including daratumumab and isatuximab. Other anti-CD38 antibodies are discussed in, e.g., WO 2005/103083, WO 2006/125640, WO 2007/042309, and WO 2008/047242.

### Efficacy and Monitoring

The methods discussed in the present disclosure may further comprise monitoring efficacy parameters associated with effects of the BCMA inhibitor, in combination with a CD38 inhibitor, including measurement of serum protein electrophoresis (SPEP), 24-hour urine sample for urine protein electrophoresis (UPEP), serum and urine immunofixation, serum free light chain (FLC) testing, bone marrow aspirate and/or biopsy including minimal residual disease (MRD), immunoglobulin quantification (immunoglobulin A [IgA], immunoglobulin M [IgM], immunoglobulin G [IgG], immunoglobulin D [IgD], immunoglobulin E [IgE]), plasmacytoma assessment (measurement by imaging), whole body imaging, plasmacytoma biopsy, and survival status. The methods may further include cytokine release syndrome (CRS) monitoring and management to evaluate efficacy and safety within individual subjects or populations of subjects.

CRS has been observed in connection with the administration of anti-BCMA x CD3 antibodies. Corticosteroids or anti-IL-6 pathway therapies (e.g., sarilumab or tocilizumab) may be utilized in the management of CRS as discussed herein. Dexamethasone or other premedications (e.g., an anti-histamine or acetaminophen) may also be utilized in the management of CRS.

The efficacy analysis can include measuring any of the following:
Objective response rate (ORR) is defined as the proportion of patients with best overall response of PR or better as per the IMWG criteria.
Duration of response (DOR) is defined for responders (patients with a best overall response of PR or better). It is the time from the date of the first documented response until the first date of PD or death due to any cause, whichever occurs first. If a patient has not progressed or died by the analysis cutoff date, DOR will be censored at the time of the last adequate tumor assessment on or prior to the cutoff date.
Progression-free survival (PFS) is defined as the time from the start of study drug regimen until the first date of PD, or death due to any cause, whichever occurs first. If a patient has not progressed or died by the analysis cutoff date, PFS will be censored at the time of the last adequate tumor assessment on or prior to the cutoff date.
The rate of MRD negativity is defined as the proportion of patients who have reached MRD negativity. The MRD negativity rate can be summarized using descriptive statistics, along with 2-sided 95% binomial exact CI.
Overall survival (OS) is measured from the start of study drug regimen until death due to any cause. If a patient is not known to have died at the date of the analysis cutoff, OS will be censored at the last date that the patient is documented to be alive.

### Pharmaceutical Compositions and Administration

The present disclosure includes methods which comprise administering a BCMA inhibitor (e.g., a bispecific anti-BCMA/anti-CD3 antibody or antigen-binding fragment thereof) in combination with administration of a CD38 inhibitor to a subject wherein the BCMA inhibitor and/or the CD38 inhibitor is contained within a pharmaceutical composition. The BCMA inhibitor and the CD38 inhibitor can be formulated together or separately. Preferably, the BCMA inhibitor and the CD38 inhibitor are formulated for separate administration to a subject. The pharmaceutical compositions of the disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, or by injection, and may be administered together with other biologically active agents. In some cases, the antibodies are administered subcutaneously, or subcutaneously and intravenously.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent. The injection thus prepared is preferably filled in an appropriate ampoule.

Advantageously, the pharmaceutical compositions for use described above are prepared into dosage forms in a unit dose suited to fit a dose of the active ingredients. Such dosage forms in a unit dose include, for example, a vial or a prefilled syringe. In some embodiments, a vial can contain a single dose of a therapeutic agent, or multiple doses of a single therapeutic agent or multiple therapeutic agents.

### Additional Combination Therapies

The present disclosure provides methods which comprise administering a BCMA inhibitor (e.g., a bispecific anti-BCMA/anti-CD3 antibody or antigen-binding fragment thereof described herein) in combination with a CD38 inhibitor. The present disclosure also provides methods which comprise administering a BCMA inhibitor in combination with a CD38 inhibitor, further in combination with an additional therapeutic agent. Exemplary additional therapeutic agents that may be combined with or administered in combination with a bispecific antibody or antigen-binding fragment and a CD38 inhibitor, include, e.g., an anti-tumor agent (e.g. chemotherapeutic agents including melphalan, vincristine (Oncovin), proteasome inhibitors (e.g. bortexomib), cyclophosphamide (Cytoxan), etoposide (VP-16), doxorubicin (Adriamycin), liposomal doxorubicin (Doxil), obendamustine (Treanda), hyaluronidase, or any others known to be effective in treating a plasma cell tumor in a subject). In some embodiments, the additional therapeutic agent comprises steroids. In some embodiments, the additional therapeutic agent comprises targeted therapies including thalidomide or lenalidomide, which are therapies approved to treat newly diagnosed patients. Lenalidomide, pomalidomide, panobinostat, and elotuzumab are examples of additional therapeutic agents effective for treating recurrent multiple myeloma. In certain embodiments the additional therapeutic agent is a regimen comprising radiotherapy or a stem cell transplant. In certain embodiments, the additional therapeutic agent may be an immunomodulatory agent. In certain embodiments the additional therapeutic agent may be a histone deacetylase inhibitor such as panobinostat (Farydak). In certain embodiments, the additional therapeutic agent may be a monoclonal antibody, an antibody drug conjugate, a checkpoint inhibitor, an oncolytic virus, a chimeric antigen receptor (CAR), or combinations thereof. Other agents that may be beneficially administered in combination with the bispecific antibodies or antigen-binding fragments and CD38 inhibitors of the disclosure include cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors. The combination of anti-BCMA x anti-CD3 bispecific antibodies or antigen-binding fragments and a CD38 inhibitor may also be administered as part of a therapeutic regimen comprising one or more therapeutic combinations selected from monoclonal antibodies other than those described herein, which may interact with a different antigen on the plasma cell surface, a bispecific antibody, which has one arm that binds to an antigen on the tumor cell surface and the other arm binds to an antigen on a T cell, an antibody drug conjugate, a checkpoint inhibitor, for example, one that targets, PD-1, PD-L1 or CTLA-4, or combinations thereof. In certain embodiments, the checkpoint inhibitors may be selected from PD-1 inhibitors, such as pembrolizumab (Keytruda), nivolumab (Opdivo), or cemiplimab (REGN2810). In certain embodiments, the checkpoint inhibitors may be selected from PD-L1 inhibitors, such as atezolizumab (Tecentriq), avelumab (Bavencio), or Durvalumab (Imfinzi). In certain embodiments, the checkpoint inhibitors may be selected from CTLA-4 inhibitors, such as ipilimumab (Yervoy).

The additional therapeutically active component(s) may be administered just prior to, concurrent with, or shortly after the administration of a BCMA inhibitor and/or a CD38 inhibitor. In some cases, the combination of agents are formulated separately (e.g., in different pharmaceutical compositions). In some cases, the combination of agents are formulated together (e.g., in the same pharmaceutical composition). The present disclosure also includes pharmaceutical compositions in which a BCMA inhibitor is co-formulated with a CD38 inhibitor, and with one or more of the additional therapeutically active component(s) as described elsewhere herein.

### Administration Regimens

The present disclosure includes methods comprising administering to a subject a BCMA inhibitor (e.g., a bispecific anti-BCMA x CD3 antibody or antigen-binding fragment thereof), in combination with a CD38 inhibitor.

The BCMA inhibitor may be administered at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, administration of the BCMA inhibitor is once weekly (QW). In some embodiments, administration of the BCMA inhibitor is twice weekly (2QW). In some embodiments, administration of the BCMA inhibitor is thrice weekly (3QW). In some embodiments, administration of the BCMA inhibitor is once every two weeks (Q2W). In some embodiments, administration of the BCMA inhibitor is once every three weeks (Q3W). In some embodiments, administration of the BCMA inhibitor is once every four weeks (Q4W).

The CD38 inhibitor may be administered at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, administration of the CD38 inhibitor is once weekly (QW). In some embodiments, administration of the CD38 inhibitor is twice weekly (2QW). In some embodiments, administration of the CD38 inhibitor is thrice weekly (3QW). In some embodiments, administration of the CD38 inhibitor is once every two weeks (Q2W). In some embodiments, administration of the CD38 inhibitor is once every three weeks (Q3W). In some embodiments, administration of the CD38 inhibitor is once every four weeks (Q4W).

According to certain embodiments of the present disclosure, multiple doses of a BCMA inhibitor (e.g., a bispecific anti-BCMA/anti-CD3 antibody or antigen-binding fragment thereof), or mulitple doses of a CD38 inhibitor, may be administered to a subject over a defined time course. The methods according to this aspect of the disclosure comprise administering to a subject one or more doses of a BCMA inhibitor in combination with one or more doses of a CD38 inhibitor. As used herein, “sequential administering” means that each dose of the BCMA inhibitor is administered to the subject at a different point in time, e.g., on different days separated by a predetermined interval (e.g., hours, days, weeks or months). The present disclosure includes methods which comprise sequential administration to the patient a single initial dose of a BCMA inhibitor, followed by one or more secondary doses of the BCMA inhibitor, and optionally followed by one or more tertiary doses of the BCMA inhibitor. The present disclosure includes methods which comprise sequential administration to the patient of a single initial dose of the CD38 inhibitor, followed by one or more secondary doses of the CD38 inhibitor, and optionally followed by one or more tertiary doses of the CD38 inhibitor.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose (also referred to as the “intermediate dose”; and the “tertiary doses” are the doses which are administered after the secondary doses (also referred to as the “full dose”). The initial, secondary, and tertiary doses may all contain the same amount of the BCMA inhibitor (e.g., the bispecific antibody) or the same amount of the CD38 inhibitor. In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses,” of either the BCMA inhibitor or the CD38 inhibitor, followed by subsequent doses that are administered on a less frequent basis (e.g., “maintenance doses”).

In one exemplary embodiment of the present disclosure, each secondary and/or tertiary dose is administered 1 to 10 (e.g., 1, 1.5, 2, 2.5, 3, 3.5, or 4, or more) days after the immediately preceding dose. The phrase “the immediately preceding dose,” as used herein, means, in a sequence of multiple administrations, the dose of a BCMA inhibitor and/or the dose of a CD38 inhibitor to a patient prior to the administration of the very next dose in the sequence with no intervening doses.

The methods according to this aspect of the disclosure may comprise administering to a patient any number of secondary and/or tertiary doses of a BCMA inhibitor, with any number of secondary and/or tertiary doses of a CD38 inhibitor. For example, in certain embodiments, only a single secondary dose of the BCMA inhibitor, and/or only a single secondary dose of the CD38 inhibitor, is administered to the patient. In other embodiments, two or more (e.g., 2, 3, 4, 5, 6, 7, 8, or more) secondary doses of either agent, or both agents, are administered to the patient. Likewise, in certain embodiments, only a single tertiary dose of either agent, or both agents, is administered to the patient. In other embodiments, two or more (e.g., 2, 3, 4, 5, 6, 7, 8, or more) tertiary doses of either agent, or both agents, are administered to the patient.

In embodiments involving multiple secondary doses of both agents, each secondary dose may be administered at the same frequency as the other secondary doses. For example, each secondary dose of either the BCMA inhibitor or the CD38 inhibitor may be administered to the patient 1, 2 or 3 weeks (e.g., 1 week) after the immediately preceding dose. Similarly, in embodiments involving multiple tertiary doses, each tertiary dose may be administered at the same frequency as the other tertiary doses. For example, each tertiary dose may be administered to the patient 1 to 4 weeks (e.g., 1 week, 2 weeks, or 4 weeks) after the immediately preceding dose. Alternatively, the frequency at which the secondary and/or tertiary doses are administered to a patient can vary over the course of the treatment regimen (e.g., the tertiary or “full” doses may begin with weekly administration, and then be modified to once every two weeks (Q2W) and/or once every four weeks (Q4W) administration. The frequency of administration may also be adjusted during the course of treatment by a physician depending on the needs of the individual patient following clinical examination.

In embodiments of the methods discussed herein, the BCMA inhibitor, and/or the CD38 inhibitor, may be administered for up to 99 weeks or more (e.g., once weekly, once every other week, or once every four weeks). In some cases, the BCMA inhibitor and/or the CD38 inhibitor may be administered for 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, or 100 or more weeks at one or more doses as discussed herein.

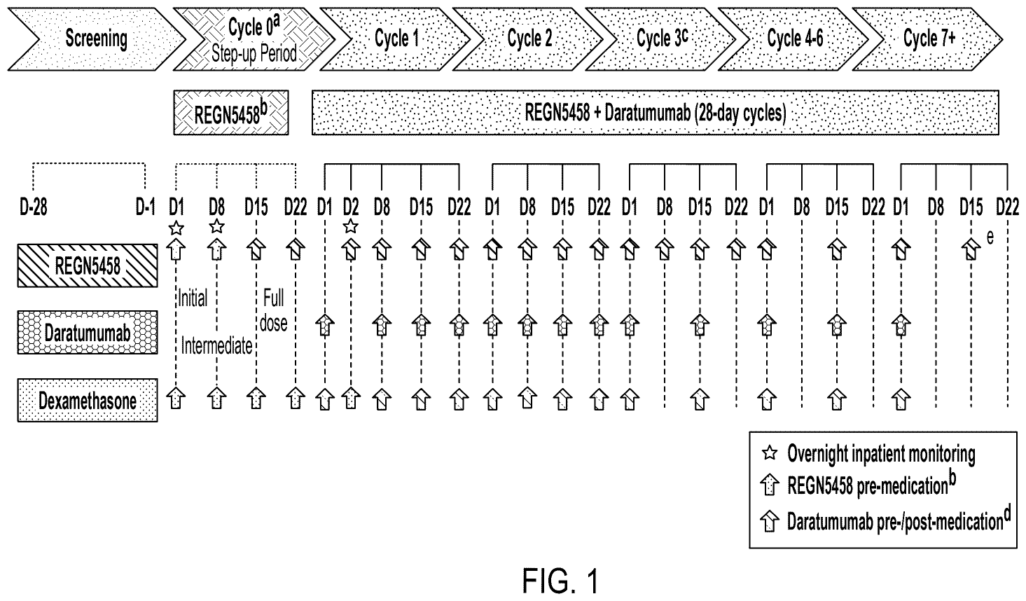

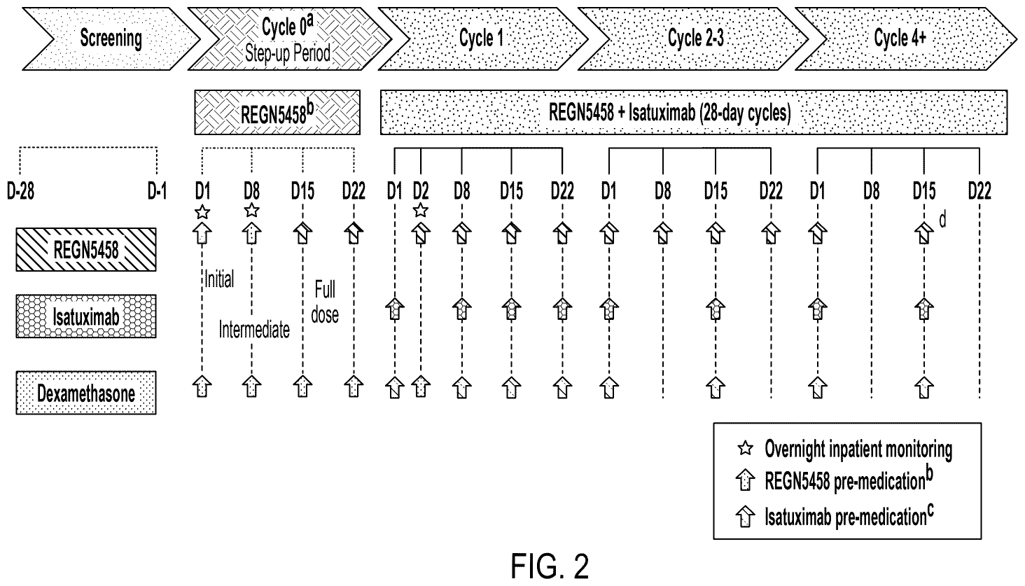

In embodiments of the methods discussed herein, the BCMA inhibitor (e.g., a bispecific antibody or antigen-binding fragment thereof), and daratumumab, an exemplary CD38 inhibitor, are administered in a dosing regimen as shown in FIG. 1. In embodiments of the methods discussed herein, the bispecific antibody or antigen-binding fragment thereof, and isatuximab, an exemplary CD38 inhibitor, are administered in a dosing regimen as shown in FIG. 2.

In some embodiments of the methods discussed herein, a premedication is used prior to administration of the combination of a BCMA inhibitor and CD38 inhibitor. For example, premedication such as an antihistamine, acetaminophen, IL-6R inhibitor, and/or dexamethosone may be used prior to administration of the bispecific antibody and/or the CD38 inhibitor. An exemplary premedication dosing schedule for use with an anti-BCMA x CD3 antibody in combination with a CD38 inhibitor is summarized in Tables 2A and 2B for isatuximab and daratumumab, respectively.

TABLE 2A
Exemplary Premedication Dosing Schedule

| Dexamethasone Administration Schedule | Cycle 1 | Cycle 2+ |
|---|---|---|
| Day 1 | | |
| Premedication 1 to 3 hours prior to | 20 mg IV | 20 mg IV |
| isatuximab infusion | | or PO |
| Day 2 | | |
| Premedication 1 to 3 hours prior to | 20 mg IV | |
| REGN5458 infusion | | |
| Day 8 | | |
| Premedication 1 to 3 hours prior to | 40 mg IV | |
| isatuximab infusion | | |
| Day 15 | | |
| Premedication 1 to 3 hours prior to | 40 mg IV | 20 mg IV |
| isatuximab infusion | | or PO |
| Day 22 | | |
| Premedication 1 to 3 hours prior to | 40 mg IV | |
| isatuximab infusion | | |

TABLE 2B
Exemplary Premedication Dosing Schedule

| Dexamethasone | Cycle | Cycle | Cycles | Cycle |
|---|---|---|---|---|
| Administration Schedule | 1 | 2 | 3 to 6 | 7+ |

Day 1

| | | | | |
|---|---|---|---|---|
| Premedication 1 to 3 hours | 20 mg | 20 mg | 20 mg | 20 mg |
| prior to daratumumab infusion | IV | IV | IV | IV |

Day 2

| | | | | |
|---|---|---|---|---|
| Premedication 1 to 3 hours | 20 mg | | | |
| prior to REGN5458 infusion | IV | | | |
| Post-medication approximately | | 4 mg | 4 mg | 4 mg |
| 24 hours after daratumumab EOI | | PO | PO | PO |

Day 3

| | | | |
|---|---|---|---|
| Post-medication approximately | 4 mg | 4 mg | 4 mg |
| 48 hours after daratumumab EOI | PO | PO | PO |

Day 8

| | | |
|---|---|---|
| Premedication 1 to 3 hours | 20 mg | 20 mg |
| prior to daratumumab infusion | IV | IV or |
| | | PO |

Day 9

| | | |
|---|---|---|
| Post-medication approximately | 20 mg | 4 mg |
| 24 hours after daratumumab EOI | IV or | PO |
| | PO | |

Day 10

| | |
|---|---|
| Post-medication approximately | 4 mg |
| 48 hours after daratumumab EOI | PO |

Day 15

| | | | |
|---|---|---|---|
| Premedication 1 to 3 hours | 20 mg | 20 mg | 20 mg |
| prior to daratumumab infusion | IV | IV or | IV or |
| | | PO | PO |

Day 16

| | | | |
|---|---|---|---|
| Post-medication approximately | 20 mg | 4 mg | 4 mg |
| 24 hours after daratumumab EOI | IV or | PO | PO |
| | PO | | |

Day 17

| | | |
|---|---|---|
| Post-medication approximately | 4 mg | 4 mg |
| 48 hours after daratumumab EOI | PO | PO |

Day 22

| | | |
|---|---|---|
| Premedication 1 to 3 hours | 20 mg | 20 mg |
| prior to daratumumab infusion | IV | IV or |
| | | PO |

Day 23

| | | |
|---|---|---|
| Post-medication approximately | 20 mg | 4 mg |
| 24 hours after daratumumab | IV or | PO |
| infusion | PO | |

Day 24

| | |
|---|---|
| Post-medication approximately | 4 mg PO |
| 48 hours after daratumumab | |
| infusion | |

### Dosage

The amount of BCMA inhibitor (e.g., a bispecific anti-BCMA/anti-CD3 antibody or antigen-binding fragment thereof), and/or the amount of a CD38 inhibitor administered to a subject according to the methods of the present disclosure is, generally, a therapeutically effective amount. As used herein, the phrase “therapeutically effective amount” means an amount of BCMA inhibitor (a bispecific anti-BCMA/anti-CD3 antibody or antigen-binding fragment thereof) or an amount of a CD38 inhibitor (e.g., an anti-CD38 antibody such as daratumumab or isatuximab), that results in one or more of: (a) treatment of relapsed or refractory multiple myeloma in a subject; and/or (b) enhanced survival of a multiple myeloma patient, as compared to an untreated control (or control population), or a control (or control population) treated with other standard of care agents.

In the case of a bispecific anti-BCMA/anti-CD3 antibody or antigen-binding fragment thereof, a therapeutically effective amount can be from about 1 milligram (mg) to about 1000 mg, e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, aobut 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 310 mg, about 320 mg, about 330 mg, about 340 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of the bispecific anti-BCMA/anti-CD3 antibody or antigen-binding fragment thereof. In certain embodiments, the bispecific antibodies or antigen-binding fragments are administered at doses of 5 mg, 25 mg, 50 mg, 100 mg, 150 mg, and/or 200 mg in a dosing regimen to the subject to treat relapsed or refactory multiple myeloma. In certain embodiments, the bispecific antibodies or antigen-binding fragments are administered at doses of 5 mg, 25 mg, and 50 mg in a dosing regimen to the subject to treat relapsed or refractory multiple myeloma. In certain embodiments, the bispecific antibodies or antigen-binding fragments are administered at doses of 5 mg, 25 mg, and 100 mg in a dosing regimen to the subject to treat relapsed or refractory multiple myeloma. In certain embodiments, the bispecific antibodies or antigen-binding fragments are administered at doses of 5 mg, 25 mg, and 200 mg in a dosing regimen to the subject to treat relapsed or refractory multiple myeloma.

A CD38 inhibitor, e.g., daratumumab, can be administered at a dose (e.g., a therapeutically effective amount) in a range from about 1 mg/kg body weight to 35 mg/kg, e.g., about 1 mg mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 15.1 mg/kg, about 15.2 mg/kg, about 15.3 mg/kg, about 15.4 mg/kg, about 15.5 mg/kg, about 15.6 mg/kg, about 15.7 mg/kg, about 15.8 mg/kg, about 15.9 mg/kg, about 16 mg, about 16.1 mg/kg, about 16.2 mg/kg, about 16.3 mg/kg, about 16.4 mg/kg, about 16.5 mg/kg, about 16.6 mg/kg, about 16.7 mg/kg, about 16.8 mg/kg, about 16.9 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, or about 35 mg/kg. In certain embodiments, daratumumab is administered at a dose of 10 to 25 mg/kg in a dosing regimen to the subject to treat RRMM. In certain embodiments, daratumumab is administered at a dose of about 16 mg/kg in a dosing regimen to the subject to treat RRMM. In embodiments, these doses of CD38 inhibitor (e.g., an anti-CD38 antibody such as daratumumab) are administered intravenously.

A therapeutically effective amount of daratumumab can also be from about 1 milligram (mg) to about 3000 mg, e.g., about 1 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, about 2400 mg, about 2450 mg, about 2500 mg, about 2550 mg, about 2600 mg, about 2650 mg, about 2700 mg, about 2750 mg, about 2800 mg, about 2850 mg, about 2900 mg, about 2950 mg, or about 3000 mg of daratumumab. In certain embodiments, daratumumab is administered subcutaneouly at doses of about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500, or about 2600 mg in a dosing regimen to the subject to treat RRMM. Subcutaneous administrations of daratumumab can be accompanied by administration of hyaluronidase, for example, at a dose of about 10,000 to 50,000 units of hyaluronidase. In various embodiments, the accompanying dose of hyaluronidase is about 20,000 units, about 21,000 units, about 22,000 units, about 23,000 units, about 24,000 units, about 25,000 units, about 26,000 units, about 27,000 units, about 28,000 units, about 29,000 units, about 30,000 units, about 31,000 units, about 32,000 units, about 33,000 units, about 34,000 units, about 35,000 units, about 36,000 units, about 37,000 units, about 38,000 units, about 39,000 units, or about 40,000 units. In embodiments, these doses of CD38 inhibitor (e.g., an anti-CD38 antibody such as daratumumab) and hyaluronidase are administered subcutaneously. In some embodiments, about 1800 mg of daratumumab and about 30,000 units of hyaluronidase are administered subcutaneously to a subject in need thereof.

Some CD38 inhibitors, e.g., isatuximab, can be administered at a dose (e.g., a therapeutically effective amount) in a range of from about 1 mg/kg body weight to 20 mg/kg, e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 9.1 mg/kg, about 9.2 mg/kg, about 9.3 mg/kg, about 9.4 mg/kg, about 9.5 mg/kg, about 9.6 mg/kg, about 9.7 mg/kg, about 9.8 mg/kg, about 9.9 mg/kg, about 10 mg, about 10.1 mg/kg, about 10.2 mg/kg, about 10.3 mg/kg, about 10.4 mg/kg, about 10.5 mg/kg, about 10.6 mg/kg, about 10.7 mg/kg, about 10.8 mg/kg, about 10.9, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg. In certain embodiments, isatuximab is administered at a dose of 5 to 15 mg/kg in a dosing regimen to the subject to treat RRMM. In certain embodiments, isatuximab is administered at a dose of about 10 mg/kg in a dosing regimen to the subject to treat RRMM.

Additionally, a therapeutically effective amount of CD38 inhibitor (e.g., isatuximab) can be from about 1 milligram (mg) to about 1500 mg, e.g., about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, 300 mg, about 310 mg, about 320 mg, about 330 mg, about 340 mg, about 350 mg, about 400 mg, about 450 mg, about 500 mg, about 550 mg, about 600 mg, about 650 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1250 mg, about 1300 mg, about 1350 mg, about 1400 mg, about 1450 mg, or about 1500 mg of the CD38 inhibitor. In certain embodiments, isatuximab is administered at doses of about 650 mg, about 700 mg, about 750 mg, about 800 mg, about 850 mg, about 900 mg, or about 950 mg in a dosing regimen to the subject in need thereof to treat RRMM.

Exemplary dosage regimes and dosing schedules for daratumumab and isatuximab are shown in FIGS. 1 and 2, respectively.

A summary of the sequences and the corresponding SEQ ID NOs referenced herein is shown in Table 3, below.

TABLE 3
Summary of Sequences

| SEQ ID NO: | Description |
|---|---|
| 1 | Anti-BCMA Heavy Chain Variable Region |
| 2 | Anti-BCMA HCDR1 |
| 3 | Anti-BCMA HCDR2 |
| 4 | Anti-BCMA HCDR3 |
| 5 | Anti-CD3 Heavy Chain Variable Region (REGN5458) |
| 6 | Anti-CD3 HCDR1 (REGN5458) |
| 7 | Anti-CD3 HCDR2 (REGN5458) |
| 8 | Anti-CD3 HCDR3 (REGN5458) |
| 9 | Anti-CD3 Heavy Chain Variable Region (REGN5459) |
| 10 | Anti-CD3 HCDR1 (REGN5459) |
| 11 | Anti-CD3 HCDR2 (REGN5459) |
| 12 | Anti-CD3 HCDR3 (REGN5459) |
| 13 | Anti-BCMA and Anti-CD3 Light Chain Variable Region |
| 14 | Anti-BCMA and Anti-CD3 LCDR1 |
| 15 | Anti-BCMA and Anti-CD3 LCDR2 |
| 16 | Anti-BCMA and Anti-CD3 LCDR3 |
| 17 | Anti-BCMA Heavy Chain |
| 18 | Anti-CD3 Heavy Chain (REGN5458) |
| 19 | Anti-CD3 Heavy Chain (REGN5459) |
| 20 | Anti-BCMA and Anti-CD3 Light Chain |

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Bispecific Antibodies that Bind B-Cell Maturation Antigen (BCMA) and CD3

Bispecific antibodies comprising an anti-BCMA-specific binding domain and an anti-CD3-specific binding domain were constructed using standard methodologies, wherein the anti-BCMA antigen binding domain and the anti-CD3 antigen binding domain each comprise different, distinct HCVRs paired with a common LCVR. In some instances the bispecific antibodies were constructed utilizing a heavy chain from an anti-CD3 antibody, a heavy chain from an anti-BCMA antibody, and a common light chain (see Table 4).

The bispecific antibodies created in accordance with the present Example comprise two separate antigen-binding domains (i.e., binding arms). The first antigen-binding domain comprises a heavy chain variable region derived from an anti-BCMA antibody (“BCMA-VH”), and the second antigen-binding domain comprises a heavy chain variable region derived from an anti-CD3 antibody (“CD3-VH”). Both the anti-BCMA and the anti-CD3 binding domains share a common light chain. The BCMA-VH/CD3-VH pairing creates antigen-binding domains that specifically recognize CD3 on T cells and BCMA on tumor cells.

A summary of the component parts of the antigen-binding domains of the anti-BCMA x CD3 bispecific antibodies constructed is set forth in Table 4. The corresponding CDR sequences and full-length heavy and light chain sequences are identified in Table 3.

TABLE 4
Summary of Component Parts of Anti-
BCMA × Anti-CD3 Bispecific Antibodies

| | Anti-BCMA | Anti-CD3 | |
|---|---|---|---|
| | Antigen-Binding | Antigen-Binding | |
| Bispecific | Domain | Domain | Common |
| Antibody | Heavy Chain | Heavy Chain | Light Chain |
| Identifier | Variable Region | Variable Region | Variable Region |
| REGN5458 | SEQ ID NO: 1 | SEQ ID NO: 5 | SEQ ID NO: 13 |
| (linvoseltamab) | | | |
| REGN5459 | SEQ ID NO: 1 | SEQ ID NO: 9 | SEQ ID NO: 13 |

### Example 2: Methods of Treating Relapsed or Refractory Multiple Myeloma with Anti-BCMA x Anti-CD3 Bispecific Antibodies in Combination with an Anti-CD38 Antibody (Daratumumab)

This study evaluates the safety and efficacy of a combination of REGN5458 (a bispecific anti-BCMA x CD3 antibody) and daratumumab (a CD38 inhibitor) in subjects with relapsed/refractory multiple myeloma.

Objectives: Both primary and secondary objectives will be explored.

The primary objective of this study is to assess the safety and tolerability and identify the recommended phase 2 dose regimen (RP2D(s)) of REGN5458 in combination with daratumumab.

The secondary objectives of the study are:
To assess the preliminary anti-tumor activity by International Myeloma Working Group (IMWG) criteria.
To measure the depth and durability of response
To evaluate the pharmacokinetics (PK) properties of REGN5458 when given in combination with a CD38 inhibitor (daratumumab)
To evaluate immunogenicity of REGN5458 when given in combination with a CD38 inhibitor
To evaluate the overall survival (OS)

Study Design: This is a phase 1b, open-label, study of the safety, tolerability, preliminary anti-tumor activity, and pharmacokinetics (PK) of REGN5458 (anti-B-cell maturation antigen [BCMA] x anti-cluster of differentiation 3 [CD3] bispecific monoclonal antibody [mAb]) in combination with daratumumab, an anti-CD38 antibody, for patients with relapsed/refractory multiple myeloma (RRMM). The study includes a dose finding portion and a dose expansion portion. Patients will receive REGN5458 in combination with daratumumab until disease progression or any other criterion for discontinuation is met.

Study Duration: The total duration of study participation for each patient may vary. The study consists of three periods: (1) screening period, (2) treatment period, and (3) follow-up period. The screening period lasts for up to 28 days and ends at day-1 before the planned first dose (initial step-up dose on cycle 0, day 1). The treatment period is of variable duration and will start with cycle 0 (step-up period) consisting of incremental doses of REGN5458 monotherapy. The patient will then receive cycles of combination treatment with REGN5458 plus daratumumab from cycle 1 onward. The end of the treatment period is defined as the day when the last dose of treatment is administered. The follow-up period includes the post-treatment follow-up period and the survival follow-up period. In the post-treatment follow-up period, patients will be followed for safety and efficacy. For safety follow-up, this will begin following the last dose of the study regimen and will include 30-day, W8 (˜60 day), and W12 (˜90 day) visits following the last dose of the study regimen, or on the day before the start of a new treatment for MM, whichever is earlier. For efficacy follow-up, for all patients who discontinue the study regimen for any reason other than disease progression, start of subsequent non-protocol anti-MM therapy, or death, will continue to undergo response assessments following the last dose of study regimen at the 30-day and W8 (˜60 day) visits followed by every 8 weeks (Q8W) thereafter until the time of disease progression, death, start of a subsequent non-protocol anti-MM therapy, or patient withdrawal of consent for follow-up, whichever is earlier. For the survival follow-up period, all patients who discontinue the study regimen or post-treatment follow-up due to disease progression or start of subsequent non-protocol myeloma therapy (whichever is earlier), will be followed every 12 weeks (Q12W) after the end of study for survival status until death, loss to follow-up, patient withdrawal of consent, or study termination by the sponsor, whichever is earlier. Patients may be followed remotely for survival information.

Study Population: Up to 44 patients will be enrolled to reach the target of 20 patients treated at the selected dose level. An additional 20 patients may be enrolled at the selected dose level to further evaluate safety and efficacy.

The study population includes patients with RRMM. Each patient must have RRMM with progression following at least three lines of therapy or at least two lines of therapy and either (1) prior exposure to at least one anti-CD38 antibody, one immunodulatory imide drug (IMiD) and one proteasome inhibitor, or (2) double refractory to one proteasome inhibitor and one IMiD or the combination of one proteasome inhibitor and one IMiD.

Refractory disease is defined as progression during treatment or within 60 days after completion of therapy, or <25% response to therapy. Prior treatment with daratumumab is allowed if previously tolerated. However, patients enrolled in the expansion portion cannot be refractory to an anti-CD38 antibody-containing regimen. In addition, all patients must have at least a 6-month washout from prior anti-CD38 antibody therapy.

Inclusion Criteria-A patient must meet the following criteria to be eligible for inclusion in the study:

1. Age 18 years or greater.

2. Eastern Cooperative Oncology Group (ECOG) performance status≤1:
a. Individual cases of patients with ECOG 2 performance status, whose ECOG status is expected to improve because of effective therapy, may be discussed with the medical monitor for potential enrollment.

3. Relapsed/refractory multiple myeloma with progression following:
at least three lines of therapy, or
at least two lines of therapy and either prior exposure to at least one anti-CD38 antibody, one IMid, and one proteasome inhibitor, or, double-refractory to one proteasome inhibitor and one IMiD, or the combination thereof.

Prior treatment with daratumumab is allowed if previously tolerated. However, patients enrolled in the expansion portion cannot be refractory to an anti-CD38 antibody containing regimen. In addition, all patients must have at least a 6-month washout from prior anti-CD38 antibody therapy.

4. Patients must have the following for response assessment as per the IMWG consensus criteria:
Measurable disease defined as 1 or more of the following:
   a. Serum M-protein ≥0.5 g/dL,
   b. Urine M-protein ≥200 mg/24-hr, and/or
   c. FLC assay with involved FLC level >10 mg/dl with an abnormal serum FLC ratio

The preferred method of response assessment for patients with immunoglobulin A (IgA) myeloma is serum IgA levels. To be eligible, the quantitative IgA levels should be ≥400 mg/dl to enable longitudinal assessments. Patients with nonsecretory MM are not eligible.

5. Adequate hematologic function, as defined by:
a. Platelet count ≥50×10⁹/L
A patient may not have received a platelet transfusion within 7 days of assessment in order to meet the platelet eligibility requirement.
b. Absolute neutrophil count (ANC) ≥1.0×10⁹/L
A patient may not have received granulocyte colony stimulating factor (G-CSF) within 5 days of assessment to meet the ANC eligibility requirement.
c. Hemoglobin ≥8.0 g/dL (red cell transfusions are allowed)

6. Adequate hepatic function, defined as:
a. Total bilirubin≤1.5×ULN; for patients with known Gilbert's syndrome, ≤3× the institutional ULN is permitted
b. Transaminase (ALT, AST)≤2.5×ULN

7. Serum creatinine clearance by Cockcroft-Gault: ≥30 mL/min
A patient with a creatinine clearance by Cockcroft-Gault formula who does not meet eligibility criteria may be considered for enrollment if a measured creatinine clearance (based on 24-hour urine collection or other reliable method) is ≥30 mL/min.

8. Life expectancy of at least 6 months.

9. Willing and able to comply with clinic visits and study-related procedures, including serial bone marrow evaluations according to the protocol schedule.
A bone marrow aspirate and biopsy, or other tissue infiltrated with malignant plasma cells, must be provided during screening for evaluation of BCMA levels in malignant cells, but demonstration of BCMA levels will not be required prior to enrollment.

10. Provide informed consent signed by the study patient.

Exclusion Criteria—A patient who meets any of the following criteria will be excluded from the study:

1. Diagnosis of plasma cell leukemia, primary systemic light-chain amyloidosis (excluding myeloma associated amyloidosis), Waldenström macroglobulinemia (lymphoplasmacytic lymphoma), or POEMS syndrome (polyneuropathy, organomegaly, endocrinopathy, monoclonal protein, and skin changes).

2. Patients with known MM brain lesions or meningeal involvement.

3. Treatment with any systemic anti-myeloma therapy within 5 half-lives or within 21 days prior to first administration of study drug regimen, whichever is shorter. Patients who experienced any AE from previous treatment that did not resolve to grade≤1 or previous treatment baseline are also excluded.

4. History of stem cell transplantation: (a) Allogenic stem cell transplantation within 6 months of start of study treatment. An elibigle patient must no longer require immunosuppressive medications and be without evidence of graft-versus-host disease; (b) Autologous stem cell transplantation within 12 weeks of start of study treatment.

5. Prior treatment with a T-cell based immunotherapy directed against BCMA, including BCMA bispecific antibodies and bispecific T-cell engagers (BiTEs), and BCMA CAR T cells. BCMA antibody-drug conjugates are not excluded.

6. Continuous systemic corticosteroid treatment with more than 10 mg per day of prednisone or anti-inflammatory equivalent within 72 hours of start of study drug regimen.

7. History of progressive multifocal leukoencephalopathy, neurodegenerative condition or CNS movement disorder, or patients with a history of seizure within 12 months prior to study enrollment are excluded.

8. Live or live attenuated vaccination within 28 days prior to first study drug regimen administration with a vector that has replicative potential; has received a COVID-19 vaccination within 1 week of planned start of study drug, or for which the planned COVID-19 vaccination would not be completed 1 week prior to start of study drug.

9. Another malignancy that is progressing or has required treatment in the last 3 years with the following exceptions:
a. Nonmelanoma skin cancer that has undergone potentially curative therapy.
b. In situ carcinoma that has been deemed to be effectively treated with definitive local control and with curative intent.
c. Adjuvant hormonal therapy with no evidence of disease for at least 3 years.

10. Evidence of significant concurrent disease or medical condition that could interfere with the conduct of the study or put the patient at significant risk, including but not limited to significant cardiovascular disease (e.g., New York Heart Association Class III or IV cardiac disease, myocardial infarction within the previous 6 months, unstable arrhythmias, or unstable angina) and/or significant pulmonary disease (e.g., obstructive pulmonary disease and history of symptomatic bronchospasm).

11. Cardiac ejection fraction <40% by echocardiogram (Echo) or multigated acquisition (MUGA) scan.

12. Any infection requiring hospitalization or treatment with intravenous (IV) anti-infectives within 2 weeks of first administration of study drug regimen.

13. Uncontrolled infection with human immunodeficiency virus (HIV), HBV, or hepatitis C virus (HCV) infection; or other uncontrolled infection.
a. Patients with HIV who have controlled infection (undetectable viral load and CD4 count above 350 cells/microliter either spontaneously or on a stable antiviral regimen) are permitted.
b. Patients with HBV (HepBsAg+) who have controlled infection (serum HBV DNA polymerase chain reaction [PCR] that is below the limit of detection AND receiving anti-viral therapy for hepatitis B) are permitted.
c. Patients who are HCV antibody positive (HCV Ab+) who have controlled infection (undetectable HCV RNA by PCR either spontaneously or in response to a successful prior course of anti-HCV therapy) are permitted.

14. History of severe allergic reaction attributed to compounds with a similar chemical or biologic composition as that of the study drug regimen or excipient. A severe allergic reaction is defined for this purpose as that requiring hospitalization and/or treatment with epinephrine.

15. History of clinically significant cardiovascular, respiratory, hepatic, renal, gastrointestinal, endocrine, hematological, psychiatric, or neurological disease, as assessed by the investigator that may confound the results of the study or poses an additional risk to the patient by study participation.

16. Known hypersensitivity to both allopurinol and rasburicase.

17. Women of childbearing potential (WOCBP) with a positive serum beta-human chorionic gonadotropin (β-hCG) pregnancy test are ineligible for this study.

18. Pregnant or breastfeeding women.

Treatment(s): REGN5458 is administered by intravenous (IV) infusion at doses of 5 mg (initial), 25 mg (intermediate), and full doses. The starting full dose will be 100 mg. The full dose may be escalated up to a maximum of 200 mg or reduced to a minimum of 50 mg, in accordance with the dose finding rules. Daratumumab is administered intravenously at a dose of 16 mg/kg. Alternatively, daratumumab may be administered subcutaneously with hyaluronidase at dose of 1800 mg daratumumab and 30,000 units of hyaluronidase.

As shown in FIG. 1, a step-up monotherapy of REGN5458 administration may occur in cycle 0 prior to the administion of REGN5458 and daratumumab in combination. An exemplary step-up monotherapy period dosing schedule will include an initial dose of REGN5458 on day 1, an intermediate dose of REGN5458 on day 8, and full doses of REGN5458 on days 15 and 22 in cycle 0. In combination therapy, REGN5458 may be administered QW or Q2W, and daratumumab may be administered QW, Q2W or Q4W. For patients with a confirmed response of VGPR or better following six cycles of treatment, REGN5458 dosing frequency may be decreased to Q4W. Following two cycles of treatment, daratumumab adminstration may be changed from IV to SC for patient convenience. In any case, a given step-up dose of REGN5458 may be repeated if not adequately tolerated before proceeding to the next incremental dose.

Preliminary Results: Of the 12 patients enrolled in the study, 11 were refractory to their last line of therapy for multiple myeloma, and of the 11 patients treated with the combination of REGN5458 (at a dose of 100 mg) and daratumumab, six patients had a complete response and three patients had a very good partial response. No new safety concerns were identified and overall safety was found to be consistent with the known safety profiles of the drugs individually.

### Example 3: Methods of Treating Relapsed or Refractory Multiple Myeloma with Anti-BCMA x Anti-CD3 Bispecific Antibodies in Combination with an Anti-CD38 Antibody (Isatuximab)

This study will evaluate the safety and efficacy of a combination of REGN5458 (a bispecific anti-BCMA x CD3 antibody) and isatuximab (a CD38 inhibitor) in subjects with relapsed/refractory multiple myeloma.

Objectives: Both primary and secondary objectives will be explored.

The primary objective of this study is to assess the safety and tolerability and identify the recommended phase 2 dose regimen (RP2D(s)) of REGN5458 in combination with isatuximab.

The secondary objectives of the study are:
To assess the preliminary anti-tumor activity by International Myeloma Working Group (IMWG) criteria.
To measure the depth and durability of response
To evaluate the pharmacokinetics (PK) properties of REGN5458 when given in combination with a CD38 inhibitor (isatuximab)·
To evaluate immunogenicity of REGN5458 when given in combination with isatuximab
To evaluate the overall survival (OS)

Study Design: This is a phase 1b, open-label, study of the safety, tolerability, preliminary anti-tumor activity, and pharmacokinetics (PK) of REGN5458 (anti-B-cell maturation antigen [BCMA] x anti-cluster of differentiation 3 [CD3] bispecific monoclonal antibody [mAb]) in combination with isatuximab, a CD38 inhibitor, for patients with relapsed/refractory multiple myeloma (RRMM). The study includes a dose finding portion and a dose expansion portion. Patients will receive REGN5458 in combination with isatuximab until disease progression or any other criterion for discontinuation is met.

Study Duration: The total duration of study participation for each patient may vary. The study consists of three periods: (1) screening period, (2) treatment period, and (3) follow-up period. The screening period lasts for up to 28 days and ends at day-1 before the planned first dose (initial step-up dose on cycle 0, day 1). The treatment period is of variable duration and will start with cycle 0 (step-up period) consisting of incremental doses of REGN5458 monotherapy. The patient will then receive cycles of combination treatment with REGN5458 plus isatuximab from cycle 1 onward. The end of the treatment period is defined as the day when the last dose of treatment is administered. The follow-up period includes the post-treatment follow-up period and the survival follow-up period. In the post-treatment follow-up period, patients will be followed for safety and efficacy. For safety follow-up, this will begin following the last dose of the study regimen and will include 30-day, W8 (˜60 day), and W12 (˜90 day) visits following the last dose of the study regimen, or on the day before the start of a new treatment for MM, whichever is earlier. For efficacy follow-up, for all patients who discontinue the study regimen for any reason other than disease progression, start of subsequent non-protocol anti-MM therapy, or death, will continue to undergo response assessments following the last dose of study regimen at the 30-day and W8 (˜60 day) visits followed by every 8 weeks (Q8W) thereafter until the time of disease progression, death, start of a subsequent non-protocol anti-MM therapy, or patient withdrawal of consent for follow-up, whichever is earlier. For the survival follow-up period, all patients who discontinue the study regimen or post-treatment follow-up due to disease progression or start of subsequent non-protocol myeloma therapy (whichever is earlier), will be followed every 12 weeks (Q12W) after the end of study for survival status until death, loss to follow-up, patient withdrawal of consent, or study termination by the sponsor, whichever is earlier. Patients may be followed remotely for survival information.

Study Population: Up to 44 patients will be enrolled to reach the target of 20 patients treated at the selected dose level. An additional 20 patients may be enrolled at the selected dose level to further evaluate safety and efficacy. The study population includes patients with RRMM. For inclusion in the study, each patient must have RRMM with progression following at least three lines of therapy or at least two lines of therapy and either (1) prior exposure to at least one anti-CD38 antibody, one immunodulatory imide drug (IMiD) and one proteasome inhibitor, or (2) double refractory to one proteasome inhibitor and one IMID or the combination thereof.

Refractory disease is defined as progression during treatment or within 60 days after completion of therapy, or <25% response to therapy. Prior treatment with isatuximab is allowed if previously tolerated. Additionally, patients must undergo at least a 3-month washout following prior anti-CD38 antibody therapy before enrollment.

Inclusion Criteria-A patient must meet the following criteria to be eligible for inclusion in the study:

1. Age 18 years or greater.

2. Eastern Cooperative Oncology Group (ECOG) performance status≤1:
a. Individual cases of patients with ECOG 2 performance status, whose ECOG status is expected to improve because of effective therapy, may be discussed with the medical monitor for potential enrollment.

3. Relapsed/refractory multiple myeloma with progression following:
at least three lines of therapy, or
at least two lines of therapy and either prior exposure to at least one anti-CD38 antibody, one IMid, and one proteasome inhibitor, or, double-refractory to one proteasome inhibitor and one IMID, or the combination thereof.

Prior treatment with isatuximab is allowed if previously tolerated. Additionally, patients must undergo at least a 3-month washout following prior anti-CD38 antibody therapy before enrollment.

4. Patients must have the following for response assessment as per the IMWG consensus criteria:

Measurable disease defined as 1 or more of the following:
a. Serum M-protein ≥0.5 g/dL,
b. Urine M-protein ≥200 mg/24-hr, and/or
c. FLC assay with involved FLC level≥10 mg/dl with an abnormal serum FLC ratio

The preferred method of response assessment for patients with immunoglobulin A (IgA) myeloma is serum IgA levels. To be eligible, the quantitative IgA levels should be ≥400 mg/dl to enable longitudinal assessments. Patients with nonsecretory MM are not eligible.

5. Adequate hematologic function, as defined by:
a. Platelet count ≥50×10⁹/L

A patient may not have received a platelet transfusion within 7 days of assessment in order to meet the platelet eligibility requirement.
b. Absolute neutrophil count (ANC) ≥1.0×10⁹/L A patient may not have received granulocyte colony stimulating factor (G-CSF) within 5 days of assessment to meet the ANC eligibility requirement.
c. Hemoglobin ≥8.0 g/dL (red cell transfusions are allowed)

6. Adequate hepatic function, defined as:
a. Total bilirubin≤1.5×ULN; for patients with known Gilbert's syndrome, ≤3× the institutional ULN is permitted
b. Transaminase (ALT, AST)≤2.5×ULN

7. Serum creatinine clearance by Cockcroft-Gault: ≥30 mL/min A patient with a creatinine clearance by Cockcroft-Gault formula who does not meet eligibility criteria may be considered for enrollment if a measured creatinine clearance (based on 24-hour urine collection or other reliable method) is ≥30 mL/min.

8. Life expectancy of at least 6 months.

9. Willing and able to comply with clinic visits and study-related procedures, including serial bone marrow evaluations according to the protocol schedule.

A bone marrow aspirate and biopsy, or other tissue infiltrated with malignant plasma cells, must be provided during screening for evaluation of BCMA levels in malignant cells, but demonstration of BCMA levels will not be required prior to enrollment.

10. Provide informed consent signed by the study patient.

Exclusion Criteria-A patient who meets any of the following criteria will be excluded from the study:

1. Diagnosis of plasma cell leukemia, primary systemic light-chain amyloidosis (excluding myeloma associated amyloidosis), Waldenström macroglobulinemia (lymphoplasmacytic lymphoma), or POEMS syndrome (polyneuropathy, organomegaly, endocrinopathy, monoclonal protein, and skin changes).

2. Patients with known MM brain lesions or meningeal involvement.

3. Treatment with any systemic anti-myeloma therapy within 5 half-lives or within 21 days prior to first administration of study drug regimen, whichever is shorter. Patients who experienced any AE from previous treatment that did not resolve to grade≤1 or previous treatment baseline are also excluded.

4. History of stem cell transplantation: (a) Allogenic stem cell transplantation within 6 months of start of study treatment. An elibigle patient must no longer require immunosuppressive medications and be without evidence of graft-versus-host disease; (b) Autologous stem cell transplantation within 12 weeks of start of study treatment.

5. Prior treatment with a T-cell based immunotherapy directed against BCMA, including BCMA bispecific antibodies and bispecific T-cell engagers (BiTEs), and BCMA CAR T cells. BCMA antibody-drug conjugates are not excluded.

6. Continuous systemic corticosteroid treatment with more than 10 mg per day of prednisone or anti-inflammatory equivalent within 72 hours of start of study drug regimen.

7. History of progressive multifocal leukoencephalopathy, neurodegenerative condition or CNS movement disorder, or patients with a history of seizure within 12 months prior to study enrollment are excluded.

8. Live or live attenuated vaccination within 28 days prior to first study drug regimen administration with a vector that has replicative potential; has received a COVID-19 vaccination within 1 week of planned start of study drug, or for which the planned COVID-19 vaccination would not be completed 1 week prior to start of study drug.

9. Another malignancy that is progressing or has required treatment in the last 3 years with the following exceptions:
a. Nonmelanoma skin cancer that has undergone potentially curative therapy.
b. In situ carcinoma that has been deemed to be effectively treated with definitive local control and with curative intent.
c. Adjuvant hormonal therapy with no evidence of disease for at least 3 years.

10. Evidence of significant concurrent disease or medical condition that could interfere with the conduct of the study or put the patient at significant risk, including but not limited to significant cardiovascular disease (e.g., New York Heart Association Class Ill or IV cardiac disease, myocardial infarction within the previous 6 months, unstable arrhythmias, or unstable angina) and/or significant pulmonary disease (e.g., obstructive pulmonary disease and history of symptomatic bronchospasm).

11. Cardiac ejection fraction <40% by echocardiogram (Echo) or multigated acquisition (MUGA) scan.

12. Any infection requiring hospitalization or treatment with intravenous (IV) anti-infectives within 2 weeks of first administration of study drug regimen.

13. Uncontrolled infection with human immunodeficiency virus (HIV), HBV, or hepatitis C virus (HCV) infection; or other uncontrolled infection.
a. Patients with HIV who have controlled infection (undetectable viral load and CD4 count above 350 cells/microliter either spontaneously or on a stable antiviral regimen) are permitted.
b. Patients with HBV (HepBsAg+) who have controlled infection (serum HBV DNA polymerase chain reaction [PCR] that is below the limit of detection AND receiving anti-viral therapy for hepatitis B) are permitted.
c. Patients who are HCV antibody positive (HCV Ab+) who have controlled infection (undetectable HCV RNA by PCR either spontaneously or in response to a successful prior course of anti-HCV therapy) are permitted.

14. History of severe allergic reaction attributed to compounds with a similar chemical or biologic composition as that of the study drug regimen or excipient. A severe allergic reaction is defined for this purpose as that requiring hospitalization and/or treatment with epinephrine.

15. History of clinically significant cardiovascular, respiratory, hepatic, renal, gastrointestinal, endocrine, hematological, psychiatric, or neurological disease, as assessed by the investigator that may confound the results of the study or poses an additional risk to the patient by study participation.

16. Known hypersensitivity to both allopurinol and rasburicase.

17. Women of childbearing potential (WOCBP) with a positive serum beta-human chorionic gonadotropin (β-hCG) pregnancy test are ineligible for this study.

18. Pregnant or breastfeeding women.

Treatment(s): REGN5458 is administered by intravenous (IV) infusion at doses of 5 mg (initial), 25 mg (intermediate), and full doses. The starting full dose will be 100 mg. The full dose may be escalated up to a maximum of 200 mg or reduced to a minimum of 50 mg, in accordance with the dose finding rules. Isatuximab is administered intravenously at a dose of 10 mg/kg.

As shown in FIG. 2, a step-up monotherapy of REGN5458 administration may occur in cycle 0 prior to the administion of REGN5458 and isatuximab in combination. An exemplary step-up monotherapy period dosing schedule will include an initial dose of REGN5458 on day 1, an intermediate dose of REGN5458 on day 8, and full doses of REGN5458 on days 15 and 22 in cycle 0. In combination therapy, REGN5458 may be administered QW or Q2W, and isatuximab may be administered QW or Q2W. For patients with a confirmed response of VGPR or better following six cycles of treatment, REGN5458 dosing frequency may be decreased to Q4W. In any case, a given step-up dose of REGN5458 may be repeated if not adequately tolerated before proceeding to the next incremental dose.

Preliminary Results: Combination therapy comprising REGN5458 and isatuximab will be tolerated and will meet the efficacy objectives set forth herein.

### Example 4: Methods of Treating Relapsed or Refractory Multiple Myeloma with Anti-BCMA x Anti-CD3 Bispecific Antibodies in Combination with an Anti-CD38 Antibody (Danartumumab or Isatuximab)

Linvoseltamab, a human B-cell maturation antigen (BCMA) x CD3 bispecific antibody, demonstrated high efficacy and a generally manageable safety profile in patients with triple-class—exposed (TCE) RRMM. The present study investigates whether combining linvoseltamab with an anti-CD38 monoclonal antibody (mAb), such as daratumumab or isatuximab, may provide additional benefit to patients with RRMM. Pooled safety and efficacy results of linvoseltamab+daratumumab and lionvoseltamab+isatuximab cohorts from the dose-finding portion of the Phase 1b study discussed in Examples 2 and 3 are discussed below.

Eligible patients were aged ≥18 years with RRMM that progressed after ≥3 lines of therapy (LoTs), or ≥2 LoTs if TCE or double-class refractory (immunomodulatory drug [IMiD]+proteasome inhibitor [PI]). Prior treatment with daratumumab or isatuximab was allowed if previously tolerated and >6 months or >3 months, respectively, had elapsed since last exposure. Treatment began with linvoseltamab intravenously (IV) alone (Cycle [C] 0; 28-day cycle) as 2 step-up doses (5 mg, 25 mg) and 2 full doses (dose level [DL] 1:100 mg; DL1b: 150 mg; DL2: 200 mg) before initiation of standard dosing of daratumumab 16 mg/kg IV or isatuximab 10 mg/kg IV at C1 start. Linvoseltamab was given once weekly in C1-3, and once every 2 weeks during C4+. Dexamethasone premedication was required. Primary endpoints: dose-limiting toxicities (DLTs), treatment-emergent adverse events (TEAEs). Key secondary endpoints: objective response rate (ORR), duration of response (DOR), progression-free survival (PFS), and rate of minimal residual disease (MRD) negativity, all measured by International Myeloma Working Group criteria. The analyses reported here combine the linvoseltamab+daratumumab and linvoseltamab+isatuximab cohorts.

To date, 42 patients had enrolled with 38 patients receiving linvoseltamab+daratumumab or isatuximab and 4 patients receiving linvoseltamab only because they did not reach C1. All patients were included in the safety analysis set (DL1 [n=18; daratumumab 12, isatuximab 6], DL 1b [n=16; daratumumab 6, isatuximab 10], or DL2 [n=8; all daratumumab]) and 37 were included in the efficacy analysis set (DL1 [n=16; daratumumab 11, isatuximab 5], DL1b [n=14; daratumumab 5, isatuximab 9], or DL2 [n=7; all daratumumab]). All patients had received ≥1 PI (refractory to ≥1 PI: 76%) or ≥1 IMiD (refractory to ≥1 IMID: 86%), and 21 patients (50%) received prior daratumumab or isatuximab (refractory to daratumumab or isatuximab: 33% [daratumumab cohort, n=4; isatuximab cohort, n=10]). Median number of prior LoTs was 3 (range, 2-8), including 50% of patients with TCE and 21% with triple-class-refractory disease. Median age was 68 years (range, 42-86); male, 57%; International Staging System stage III at study entry, 12%; high-risk cytogenetics, 31%; bone marrow plasma cells ≥50%, 31%; and soft tissue plasmacytomas, 33% (extramedullary, 12%). Median duration of follow-up was 14.8 (DL1: daratumumab 21.3, isatuximab 11.3), 7.6 (DL1b: daratumumab 13.5, isatuximab 5.2), and 5.9 mos (DL2: daratumumab), with 44%, 50% and 63% of patients still receiving tx, respectively. The most common TEAEs were anemia (any Grade [Gr], 48%; Gr 3/4, 26%), neutropenia (41%; 41%), diarrhea (38%; 2%), and thrombocytopenia (38%; 17%). Cytokine release syndrome was reported in 33% of patients, all Gr 1 or 2 except 1 Gr 3 event during the step-up period that fully resolved. No patients experienced ICANS. Infections were reported in 32 patients (76%; Gr ≥3, 40% [daratumumab, n=12; isatuximab, n=5]). TEAEs led to treatment discontinuation in 3 patients in the linvoseltamab+daratumumab cohort. One DLT was observed at DL1b in the isatuximab cohort (Gr 3, EBV pharyngitis) and one DLT was observed at DL2 in the daratumumab cohort (Gr 5, CMV pneumonia). Among evaluable patients, ORR was 75% at DL1 (12/16; very good partial response or better [>VGPR] rate, 75%), 93% at DL1b (13/14; >VGPR rate, 57%), and 86% at DL2 (6/7; >VGPR rate, 86%). Fatal TEAEs occurred in 9.5% of patients (daratumumab only: DL1, n=2; DL1b, n=1; DL2, n=1) within 30 days of the last administered dose. Of 10 MRD-evaluable patients, all were MRD negative (<10-5 by clonoSEQ or EuroFlow). At 12 months, DOR rate was 79% (95% CI 52-92; median DOR, not reached [NR] [95% CI not evaluable (NE)-NE]); PFS rate was 73% (95% CI 53-86; median PFS, NR [95% CI 13.7-NE]). Pharmacokinetic analyses found linvoseltamab concentrations were not affected by the addition of daratumumab or isatuximab.

In patients with heavily pretreated RRMM, the combination of linvoseltamab+an anti-CD38 mAb is feasible, inducing a high rate of deep and durable responses, with a safety profile generally consistent with the individual drugs.

The present disclosure is not to be limited in scope by the specific embodiments described herein. Indeed, various modifications of the disclosure in addition to those described herein will become apparent to those skilled in the art from the foregoing description. Such modifications are intended to fall within the scope of the appended claims.

Sequences
SEQ ID NO: 1
EVQLVESGGGLVQPGGSLRLSCAASGFTFSNFWMTWVRQAPGKGLEWVANMNQDGSEKYYVDSVKGRFTISRD
NAKSSLYLQMNSLRAEDTAVYYCARDREYCISTSCYDDFDYWGQGTLVTVSS
SEQ ID NO: 2
GFTFSNEW
SEQ ID NO: 3
MNQDGSEK
SEQ ID NO: 4
ARDREYCISTSCYDDEDY
SEQ ID NO: 5
EVQLVESGGGLVQPGRSLRLSCAASGFTEDDYSMHWVRQAPGKGLEWVSGISWNSGSKGYADSVKGRFTISRD
NAKNSLYLQMNSLRAEDTALYYCAKYGSGYGKFYHYGLDVWGQGTTVTVSS
SEQ ID NO: 6
GFTEDDYS
SEQ ID NO: 7
ISWNSGSK
SEQ ID NO: 8
AKYGSGYGKFYHYGLDV
SEQ ID NO: 9
EVQLVESGGGLVQPGRSLRLSCAASGFTEDDYSMHWVRQAPGKGLEWVSGISWNSGSIGYADSVKGRFTISRD
NAKNSLYLQMNSLRAEDTALYYCAKYGSGYGKFYYYGMDVWGQGTTVTVSS
SEQ ID NO: 10
GFTEDDYS
SEQ ID NO: 11
ISWNSGSI
SEQ ID NO: 12
AKYGSGYGKFYYYGMDV
SEQ ID NO: 13
DIQMTQSPSSLSASVGDRVTITCRASQSISSYLNWYQQKPGKAPKLLIYAASSLQSGVPSRESGSGSGTDETL
TISSLQPEDFATYYCQQSYSTPPITFGQGTRLEIK
SEQ ID NO: 14
QSISSY
SEQ ID NO: 15
AAS
SEQ ID NO: 16
QQSYSTPPIT
SEQ ID NO: 17
EVQLVESGGGLVQPGGSLRLSCAASGFTFSNFWMTWVRQAPGKGLEWVANMNQDGSEKYYVDSVKGRFTISRD
NAKSSLYLQMNSLRAEDTAVYYCARDREYCISTSCYDDFDYWGQGTLVTVSSASTKGPSVEPLAPCSRSTSES
TAALGCLVKDYFPEPVTVSWNSGALTSGVHTFPAVLQSSGLYSLSSVVTVPSSSLGTKTYTCNVDHKPSNTKV
DKRVESKYGPPCPPCPAPPVAGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFSCSVMHEALHN
HYTQKSLSLSLGK
SEQ ID NO: 18
EVQLVESGGGLVQPGRSLRLSCAASGFTEDDYSMHWVRQAPGKGLEWVSGISWNSGSKGYADSVKGRFTISRD
NAKNSLYLQMNSLRAEDTALYYCAKYGSGYGKFYHYGLDVWGQGTTVTVSSASTKGPSVFPLAPCSRSTSEST
AALGCLVKDYFPEPVTVSWNSGALTSGVHTFPAVLQSSGLYSLSSVVTVPSSSLGTKTYTCNVDHKPSNTKVD
KRVESKYGPPCPPCPAPPVAGPSVFLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNVESCSVMHEALHNR
FTQKSLSLSPGK
SEQ ID NO: 19
EVQLVESGGGLVQPGRSLRLSCAASGFTEDDYSMHWVRQAPGKGLEWVSGISWNSGSIGYADSVKGRFTISRD
NAKNSLYLQMNSLRAEDTALYYCAKYGSGYGKFYYYGMDVWGQGTTVTVSSASTKGPSVEPLAPCSRSTSEST
AALGCLVKDYFPEPVTVSWNSGALTSGVHTFPAVLQSSGLYSLSSVVTVPSSSLGTKTYTCNVDHKPSNTKVD
KRVESKYGPPCPPCPAPPVAGPSVELFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNVFSCSVMHEALHNR
FTQKSLSLSPGK
SEQ ID NO: 20
DIQMTQSPSSLSASVGDRVTITCRASQSISSYLNWYQQKPGKAPKLLIYAASSLQSGVPSRESGSGSGTDETL
TISSLQPEDFATYYCQQSYSTPPITFGQGTRLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSENRGEC

1. A method of treating relapsed or refractory multiple myeloma in a subject in need thereof, comprising administering to the subject a B-cell maturation antigen (BCMA) inhibitor, and a CD38 inhibitor, wherein the BCMA inhibitor is (i) an antibody or antigen-binding fragment thereof that binds specifically to BCMA, (ii) a bispecific antibody or antigen-binding fragment thereof comprising a first antigen-binding domain that specifically binds BCMA, and a second antigen-binding domain that specifically binds CD3, (iii) an antibody-drug-conjugate wherein the antibody binds specifically to BCMA, or (iv) a cell comprising a chimeric antigen receptor (CAR) that binds specifically to BCMA.

2. The method of claim 1, wherein the CD38 inhibitor is an anti-CD38 antibody.

3. The method of claim 1, wherein CD38 inhibitor is daratumumab.

4. The method of claim 1, wherein CD38 inhibitor is isatuximab.

5. The method of claim 1, wherein the subject has not been treated with a prior anti-cancer therapy.

6. The method of claim 1, wherein the subject has been previously treated with an anti-cancer therapy.

7. The method of claim 6, wherein the subject has been previously treated with two lines of anti-cancer therapy.

8. The method of claim 7, wherein the subject has been previously treated with at least three lines of anti-cancer therapy.

9. The method of claim 8, wherein the anti-cancer therapy is selected from an anti-CD38 antibody, an immunomodulatory imide (IMiD) drug and a PI.

10. The method of claim 6, wherein the subject has been previously treated with a PI, an IMiD or a combination thereof.

11. The method of claim 6, wherein the subject is double refractory to a PI and an IMiD, or the combination of a PI and a IMiD.

12. The method of claim 6, wherein the relapsed or refractory multiple myeloma is:
multiple myeloma that has progressed during previous treatment;
multiple myeloma that has progressed within 60 days after completion of therapy; or
multiple myeloma that has had a less than 25% response to previous treatment.

13. The method of claim 1, wherein the subject is eligible for a stem cell transplant or has undergone a stem cell transplant.

14. The method of claim 1, wherein subject is not eligible for a stem cell transplant.

15. The method of claim 1, further comprising administration of a premedication prior to the administration of the BCMA inhibitor or the CD38 inhibitor.

16. The method of claim 15, wherein the premedication is an antihistamine, acetaminophen, dexamethasone, an IL6R inhibitor, or a combination thereof.

17. The method of claim 1, wherein the BCMA inhibitor and the CD38 inhibitor are administered in a dosing regimen comprising weekly (QW) administration of a full dose of the BCMA inhibitor and weekly (QW) administration of a dose of the CD38 inhibitor.

18. The method of claim 17, wherein the dosing regimen comprises one or more 28-day cycles, wherein the BCMA inhibitor is administered weekly during each week of the cycle, and the CD38 inhibitor is administered weekly during each week of the cycle.

19. The method of claim 18, wherein the one or more 28-day cycles comprise one 28-day cycle.

20. The method of claim 18, wherein the one or more 28-day cycles comprise two 28-day cycles.

21. The method of claim 19, wherein the dosing regimen further comprises one or more additional 28-day cycles.

22. The method of claim 21, wherein the BCMA inhibitor is administered weekly (QW) or once every two weeks (Q2W) or once every four weeks (Q4W) during the one or more additional 28-day cycles, and the CD38 inhibitor is administered once every two weeks (Q2W) or once every four weeks (Q4W) during the one or more additional 28-day cycles.

23. The method of claim 17, wherein the CD38 inhibitor is daratumumab.

24. The method of claim 17, wherein the CD38 inhibitor is isatuximab.

25. The method of claim 17, wherein the dosing regimen further comprises a step-up cycle preceding the one or more 28-day cycles, wherein the step-up cycle comprises administration of the BCMA inhibitor as monotherapy.

26. The method of claim 25, wherein the BCMA inhibitor is administered during the step-up cycle at an initial dose, at an intermediate dose, wherein the intermediate dose is greater than the initial dose, and at a full dose, wherein the full dose is greater than the intermediate dose.

27. The method of claim 26, wherein the initial dose is 0.5 mg to 10 mg.

28. The method of claim 26, wherein the initial dose is about 5 mg.

29. The method of claim 27, wherein the intermediate dose is 20 mg to 30 mg.

30. The method of claim 29, wherein the intermediate dose is about 25 mg.

31. The method of claim 29, wherein the full dose is 40 mg to 250 mg.

32. The method of claim 31, wherein the full dose is about 50 mg, about 100 mg, about 150 mg, or about 200 mg.

33. The method of claim 31, wherein the dose of the CD38 inhibitor is 10 mg/kg to 25 mg/kg.

34. The method of claim 33, wherein the dose of the CD38 inhibitor is about 16 mg/kg.

35. The method of claim 31, wherein the dose of the CD38 inhibitor is 5 mg/kg to 15 mg/kg.

36. The method of claim 35, wherein the dose of the CD38 inhibitor is about 10 mg/kg.

37. The method of claim 31, wherein the dose of the CD38 inhibitor is about 1400 mg to about 2200 mg.

38. The method of claim 37, wherein the dose of the CD38 inhibitor is about 1800 mg.

39. The method of claim 1, wherein the BCMA inhibitor and the CD38 inhibitor are administered intravenously or subcutaneously to the subject.

40. The method of claim 39, wherein the CD38 inhibitor is administered subcutaneously in combination with hyaluronidase.

41. The method of claim 40, wherein the dose of hyaluronidase is about 30,000 units.

42. The method of claim 1, wherein the BCMA inhibitor and the CD38 inhibitor are administered in separate formulations.

43. (canceled)

44. The method of claim 1, wherein the BCMA inhibitor is selected from the group consisting of AMG224, MEDI2228, 2A9-MICA, ALLO-605, alnuctamab, AMG-701, EM801, EMB-06, pacanalotamab, pavurutamab, linvoseltamab, vonsetamig, teclistamab, elranatamab, CM336, HBM7020, RO7297089, TQB2934, HPN217, ISB2001, IBI-3003, SAR445514, SIM0500, TNB-383B, WVT078, YKST02, balantamab mafodotin, ispectamab debotansine, LCAR-B38M, ciltacabtagene autoleucel, and idecabtagene vicleucel.

45. The method of claim 1, wherein the BCMA inhibitor is a bispecific antibody or antigen-binding fragment thereof comprising a first antigen-binding domain that specifically binds BCMA, and a second antigen-binding domain that specifically binds CD3.

46. The method of claim 45, wherein the first antigen-binding domain comprises three heavy chain complementarity determining regions, HCDR1, HCDR2 and HCDR3 comprised in a heavy chain variable region (HCVR) of SEQ ID NO: 1, and three light chain complementarity determining regions, LCDR1, LCDR2 and LCDR3 comprised in a light chain variable region (LCVR) of SEQ ID NO: 13.

47. The method of claim 46, wherein HCDR1, HCDR2 and HCDR3 comprise the amino acid sequences of SEQ ID NOs: 2, 3 and 4, respectively, and LCDR1, LCDR2 and LCDR3, comprise the amino acid sequences of SEQ ID NOs: 14, 15 and 16, respectively.

48. The method of claim 47, wherein the HCVR of the first antigen-binding domain comprises the amino acid sequence of SEQ ID NO: 1, and the LCVR of the first antigen-binding domain comprises the amino acid sequence of SEQ ID NO: 13.

49. The method of claim 46, wherein the second antigen-binding domain comprises three heavy chain complementarity determining regions, HCDR1, HCDR2 and HCDR3 comprised in a HCVR of SEQ ID NO: 5, and three light chain complementarity determining regions, LCDR1, LCDR2 and LCDR3 comprised in a LCVR of SEQ ID NO: 13.

50. The method of claim 49, wherein HCDR1, HCDR2 and HCDR3 comprise the amino acid sequences of SEQ ID NOs: 6, 7 and 8, respectively, and LCDR1, LCDR2 and LCDR3 comprise the amino acid sequences of SEQ ID NOs: 14, 15 and 16, respectively.

51. The method of claim 50, wherein the HCVR of the second antigen-binding domain comprises the amino acid sequence of SEQ ID NO: 5, and the LCVR of the first antigen-binding domain comprises the amino acid sequence of SEQ ID NO: 13.

52. The method of claim 49, wherein the second antigen-binding domain comprises three heavy chain complementarity determining regions, HCDR1, HCDR2 and HCDR3 comprised in a HCVR of SEQ ID NO: 9, and three light chain complementarity determining regions, LCDR1, LCDR2 and LCDR3 comprised in a LCVR of SEQ ID NO: 13.

53. The method of claim 52, wherein HCDR1, HCDR2 and HCDR3 comprise the amino acid sequences of SEQ ID NOs: 10, 11 and 12, respectively, and LCDR1, LCDR2 and LCDR3 comprise the amino acid sequences of SEQ ID NOs: 14, 15 and 16, respectively.

54. The method of claim 53, wherein the HCVR of the second antigen-binding domain comprises the amino acid sequence of SEQ ID NO: 9, and the LCVR of the first antigen-binding domain comprises the amino acid sequence of SEQ ID NO: 13.

55. The method of claim 45, wherein the bispecific antibody comprises a human IgG heavy chain constant region.

56. The method of claim 55, wherein the human IgG heavy chain constant region is isotype IgG1.

57. The method of claim 55, wherein the human IgG heavy chain constant region is isotype IgG4.

58. The method of claim 45, wherein the bispecific antibody comprises a chimeric hinge that reduces Fcγ receptor binding relative to a wild-type hinge of the same isotype.

59. The method of claim 45, wherein the bispecific antibody comprises a first heavy chain and a second heavy chain, and wherein the first heavy chain or the second heavy chain, but not both, comprises a C_{H}3 domain comprising a H435R (EU numbering) modification and a Y436F (EU numbering) modification.

60. The method of claim 45, wherein the bispecific antibody comprises a first heavy chain comprising the amino acid sequence of SEQ ID NO: 17, a second heavy chain comprising the amino acid sequence of SEQ ID NO: 18, and a common light chain paired with each of the first heavy chain and second heavy chain, respectively, comprising the amino acid sequence of SEQ ID NO: 20.

61. The method of claim 45, wherein the bispecific antibody comprises a first heavy chain comprising the amino acid sequence of SEQ ID NO: 17, a second heavy chain comprising the amino acid sequence of SEQ ID NO: 19, and a common light chain paired with each of the first heavy chain and second heavy chain, respectively, comprising the amino acid sequence of SEQ ID NO: 20.

62. The method of claim 45, wherein the bispecific antibody consists of two heavy chains and two lights chains interconnected by disulfide bonds, wherein each heavy chain comprises a HCVR and C_{H}1, C_{H}2 and C_{H}3 heavy chain constant domains, and each light chain comprises a LCVR and a light chain constant (LC) domain.

63. The method of claim 1, wherein the BCMA inhibitor is linvoseltamab or vonsetamig.

64. The method of claim 1, further comprising administering a third therapeutic agent or therapeutic regimen.

65. The method of claim 64, wherein the third therapeutic agent or therapeutic regimen comprises a chemotherapeutic drug, a DNA alkylator, an immunomodulator, a second proteasome inhibitor, a histone deacetylase inhibitor, radiotherapy, a stem cell transplant, a different bispecific antibody that interacts with a different tumor cell surface antigen and a T cell or immune cell antigen, an antibody drug conjugate, a chimeric antigen receptor (CAR), a PD-1 antagonist, a PD-L1 antagonist, a CTLA-4 inhibitor, a LAG3 inhibitor, a CD28 agonist, an oncolytic virus, a cytokine, an IL4R inhibitor, an IL6R inhibitor, ILIR inhibitor, IL2, IL12, IL15, IL23, or combinations thereof.
